(19) United States
(12) Patent Application Publication
(10) Pub. No.: US 2009/0004485 A1
(43) Pub. Date: Jan. 1, 2009

(54) 6-MEMBER RING STRUCTURE USED IN ELECTROLUMINESCENT DEVICES

(76) Inventors: Shiying Zheng, Webster, NY (US); Liang-Sheng Liao, Rochester, NY (US)

Correspondence Address: Frank Pincelli;Patent Legal Staff, Eastman Kodak Company, 343 State Street, Rochester, NY, 14650-2201, US

(21) Appl. No.: 11/769,189

(22) Filed: Jun. 27, 2007

(51) Int. Cl. C08G 73/06; B32B 9/00; C07C 309/63; C08G 77/04; C08G 79/06; C08G 75/00; C07D 311/02

(52) U.S. Cl. 428/446; 428/411.1; 528/380; 528/398; 528/40; 528/403; 528/417; 528/423; 549/213; 558/46

(57) ABSTRACT

An organic material having a 6-member ring structure represented by the following formulae (I),

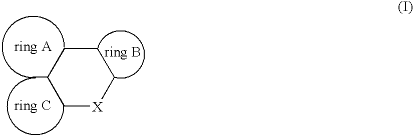

wherein:
ring A, ring B, and ring C each include substituted or un-substituted aromatic rings comprising 6 to 60 carbon atoms, or substituted or un-substituted heteroaromatic rings comprising 4 to 60 carbon atoms, and ring A and ring C form a fused aromatic or heteroaromatic structure; X is a carbon atom, a nitrogen atom, a sulfur atom, a silicon atom, an oxygen atom, a phosphorus atom, a selenium atom, or a germanium atom.

### FIELD OF THE INVENTION

The present invention relates to compounds containing a 6-member ring structure and their use in an electrical-optical device such as an organic electroluminescent (EL) device.

### BACKGROUND OF THE INVENTION

There is a great need for large area solid state light sources for a series of applications, especially in the field of display elements and lighting engineering. The demands can not be fully satisfactorily met by any of the existing technologies. Electroluminescent device such as light emitting diodes (LED) represents an alternative to conventional display and lighting elements. Electroluminescent devices are opto-electronic devices where light emission is produced in response to an electrical current through the device. The physical model for EL is the radiative recombination of electrons and holes. Both organic and inorganic materials have been used for the fabrication of LEDs. Inorganic materials such as ZnS/Sn, Ga/Bs, Ga/As have been used in semiconductor lasers, small area displays, LED lamps, etc. However, the drawbacks of inorganic materials include difficulties to process and to obtain large surface areas and efficient blue light.

Organic materials offer several advantages over inorganic materials for LEDs, such as simpler manufacturing, low operating voltages, the possibility of producing large area and full-color displays. Conjugated polymers such as poly(phenylvinylene) (PPV) were first introduced as EL materials by Burroughes et al in 1990 (Burroughes, J. H. Nature 1990, 347, 539-41). Tremendous progress has been made since then to improve the stability, efficiency, and durability of polymeric LEDs (Bernius, M. T. et al, Adv. Mater. 2000, 12, 1737). Organic LED (OLED) represents an alternative to the well-established display technologies based on cathode-ray tubes and liquid crystal displays (LCDs), especially for large area displays. OLED has been demonstrated to be brighter, thinner, lighter, and faster than LCDs. Moreover it requires less power to operate, offers higher contrast and wide viewing angle (>165 degree), and has great potential to be cheaper to manufacture, especially the solution processable polymer-based LEDs (PLED).

The OLED technology has stimulated intensive research activities across all disciplines. Currently, great efforts in materials research have been focused on novel materials for full-color flexible displays. Full-color displays require three basic colors, red, green and blue, and flexible substrates require low temperature and easy processing of the organic materials. PLED devices show great promise in meeting both requirements, since the emission color can be tailored by modulation of the chemical structures and the solution processing allows for micro-patterning of the fine multicolor pixels via inkjet printing technique (Yang, Y. et al, J. Mater. Sci.: Mater. Elecron., 2000, 11, 89). However, processable, stable, and efficient blue light emitting organic materials are still highly desirable to meet the challenge. Blue light requires wide energy band. With blue light emitting polymers as primary materials, it is possible to produce other colors by a downhill energy transfer process. For instance, a green or red EL emission can be obtained by doping a blue EL host material with a small amount of green or red luminescent material.

### SUMMARY OF THE INVENTION

It is an object of the present invention to provide novel highly efficient luminescent materials for stable EL device.

It is another object of the present invention to provide wide energy band gap luminescent materials.

It is another object of the present invention to provide novel solution processable materials for easy processing.

These objects are achieved by providing the following organic materials for an organic electroluminescent device. The organic materials comprise compounds having a 6-member ring structure represented by the following formulae (I),

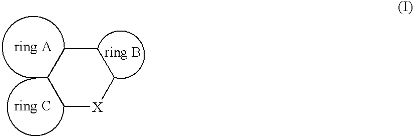

wherein:
ring A, ring B, and ring C each include substituted or un-substituted aromatic rings comprising 6 to 60 carbon atoms, or substituted or un-substituted heteroaromatic rings comprising 4 to 60 carbon atoms, and ring A and ring C form a fused aromatic or heteroaromatic structure; X is a carbon atom, a nitrogen atom, a sulfur atom, a silicon atom, an oxygen atom, a phosphorus atom, a selenium atom, or a germanium atom.

The present invention provides organic luminescent materials with a number of advantages that include excellent solubility and thermal stability, good color tunability, high efficiency, low driving voltage, and enhanced electron and/or hole transport ability.

### BRIEF DESCRIPTION OF THE DRAWINGS

FIG. 1 illustrates in cross-section a basic structure of an EL device;

FIG. 2 illustrates the absorption (AB) and photoluminescence (PL) spectra of polymer 102 in solution and thin film;

FIG. 3 illustrates the EL spectra of the EL device fabricated from polymer 102: ITO/PEDOT/polymer 102/CsF/Mg:Ag; and

FIG. 4 illustrates the voltage-current density and voltage-luminance of the EL device fabricated from polymer 102.

### DETAILED DESCRIPTION OF THE INVENTION

The present invention provides highly efficient organic light-emitting materials including compounds having a 6-member ring structure with good color tunability, excellent solubility and thermal stability, high efficiency, and enhanced electron and/or hole transport ability. The 6-member ring structure is represented by formula (I), wherein ring A, ring B, and ring C each include substituted or un-substituted aromatic rings comprising 6 to 60 carbon atoms, or substituted or un-substituted heteroaromatic rings comprising 4 to 60 carbon atoms, and ring A and ring C form a fused aromatic or heteroaromatic structure; X is a carbon atom, a nitrogen atom, a sulfur atom, a silicon atom, an oxygen atom, a phosphorus atom, a selenium atom, or a germanium atom.

In some embodiments ring A, ring B, and ring C are all 6-member aromatic or heteroaromatic rings, or all are 5-member heteroaromatic rings, or one is a 6-member aromatic or heteroaromatic ring and the other two are 5-member ring heteroaromatic ring, or two are 6-member aromatic or heteroaromatic rings and the third ring is a 5-member heteroaromatic ring. The structures of these embodiments are represented by the following formulae (Ia)-(Ih):

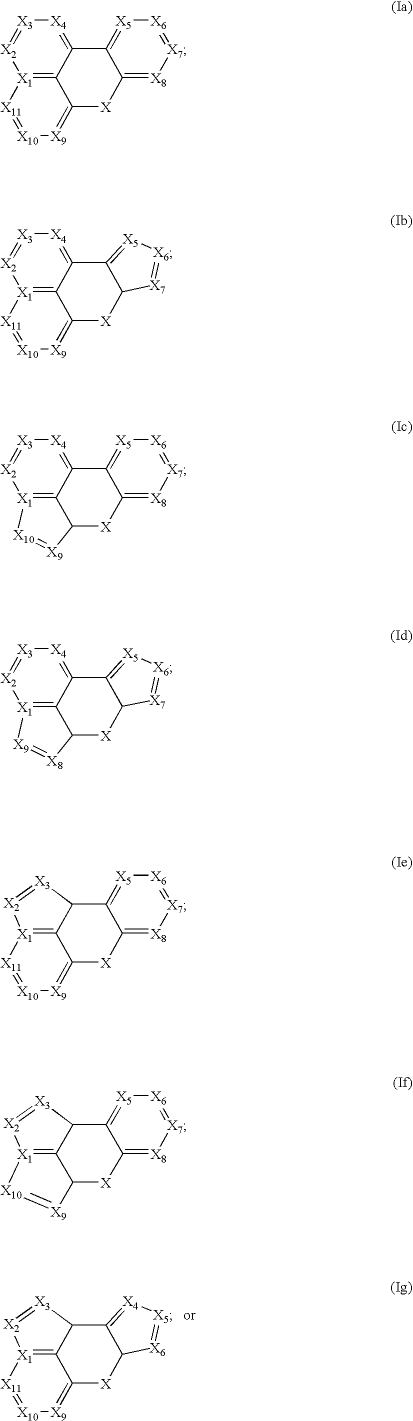

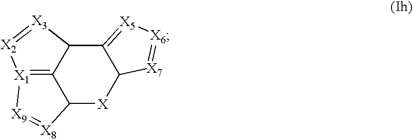

wherein: X₁, X₂, X₃, X₄, X₅, X₆, X₇, X₈, X₉, X₁₀, and X₁₁ each include a moiety containing CH, N, S, or O. For example, ring A, ring B, and ring C are independently 5-member or 6-member aromatic or heteroaromatic rings, and include but are limited to benzene, thiophene, pyridine, furan, perylene, anthracene, naphthalene, pyrrole, phenanthrene, pyrene, tetracene, pentacene, triphenylene, quinoline, and benzo[a]pyrene. X is a carbon atom, a nitrogen atom, a sulfur atom, a silicon atom, an oxygen atom, a phosphorus atom, a selenium atom, or a germanium atom. In preferred embodiments, X is a carbon atom, a nitrogen atom, a sulfur atom, or an oxygen atom. In more preferred embodiments, X is a carbon atom, a nitrogen atom, or an oxygen atom.

The organic materials comprising the 6-member ring structure include organic molecular compounds, oligomers, dendrimer, linear polymers, hyperbranched polymers and ladder polymers, and can be used in a combination of two or more thereof. The organic materials comprising the 6-member ring structure can be emissive and used as emissive materials in emissive layer in an OLED device, or as host, or as dopant in emissive layer in an OLED device. Or the organic materials can be used as charge transport layer, or both as emissive materials and charge transport materials. The organic materials can also be used in other electrical-optical devices such as organic thin film transistors and solar cells.

Organic molecular compounds comprising the 6-member ring structure are represented by formula (II)

(Y₁)y₁-formula (I)-(Y₂)y₂ (II)

wherein Y₁ and Y₂ each represent a substituted or unsubstituted alkyl, alkenyl, alkynyl, aryl, or heteroaryl or alkoxy of 1 to 60 carbons, and y₁ and y₂ are integers from 0 to 6.

Polymers comprising the 6-member ring structure are represented by repeating units of formula (ITT) which comprise the 6-member ring structure as part of the polymer main chain and repeating units of formula (IV) which comprise the 6-member ring structure as part of the polymer side chain.

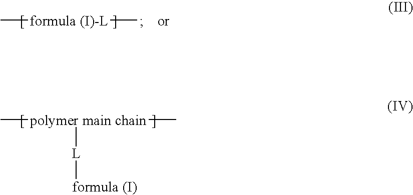

wherein:

L is a direct bond or a carbon linking group having 1 to 40 carbon atoms or a non-carbon linking group having 0 to 40 carbon atoms.

Incorporating Y₁, Y₂, and L into the compounds including the 6-member ring structure represented by formula (II), (III), and (IV) can further improve solubility, or electron or hole transporting mobility, or finely tune the emission color.

When L is a linking group, it includes but is not limited to the following groups:

### Group I:

L is a carbon-carbon linking group:

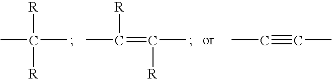

wherein R is hydrogen, alkyl, alkynyl, alkoxy, or alkenyl group containing 1 to 40 carbon atoms; aryl or substituted aryl containing 6 to 60 carbon atoms; or heteroaryl or substituted heteroaryl containing 4 to 60 carbons; or F, Cl, or Br; or a cyano, or a nitro group;

### Group II:

L is an ether or thioether linking group:

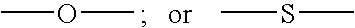

### Group III:

L is an ester linking group:

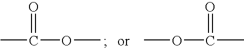

### Group IV:

L is an anhydride linking group:

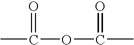

### Group V:

L is a carbonate linking group:

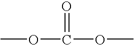

### Group VI:

L is a sulfone or sulfine linking group:

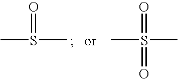

### Group VII:

L is an amine linking groups:

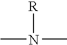

wherein R is defined as above.

### Group VIII:

L is an amide linking group:

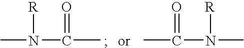

### Group IX:

L is a urea linking group:

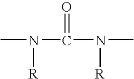

### Group X:

L is an aryl or heteroaryl linking group:

Arₙ

wherein Ar is an aryl or substituted aryl group containing 6 to 60 carbon atoms; or heteroaryl or substituted heteroaryl containing 4 to 60 carbons; n is an integer from 1 to 6.

### Group XI:

L is silyl linking group:

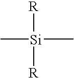

L is phosphorus linking group:

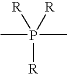

L can be one or the combination of more than one of the above groups.

Y₁ and Y₂ represent substituted or unsubstituted alkyl, alkenyl, alkynyl, aryl, heteroaryl or alkoxy groups of 1 to 60 carbon atoms, and can be the same or different.

Alkyl, alkenyl, alkynyl, and alkoxy groups contain 1 to 40 carbon atoms;

Substituted or unsubstituted aryl groups contain 6 to 60 carbon atoms which include phenyl, biphenyl, naphthyl, anthracene, fluorene, phenanthrene, spirophenyl, perylene, or pyrene groups;

Substituted or unsubstituted heteroaryl groups contain 4 to 60 carbon atoms which include pyridine, thiophene, pyrrole, bithiophene, furan, benzofuran, benzimidazole, benzoxazole, quinoxaline, phenylquinoline, dipheyloxadizaole, carbazole, and quinoline.

All the substituents mentioned above include but are not limited to alkyl or alkoxy groups containing 1 to 40 carbon atoms, aryl or substituted aryl containing 6 to 60 carbon atoms; or heteroaryl or substituted heteroaryl containing 4 to 60 carbons; or F, Cl, or Br; or a cyano group; or a nitro group.

The following structures constitute specific examples of Y₁, Y₂, and linking group L:

—(CH₂)m-
wherein: m is an integer from 1 to 6;

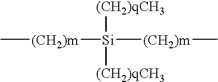

wherein: q is an integer from 0 to 12;

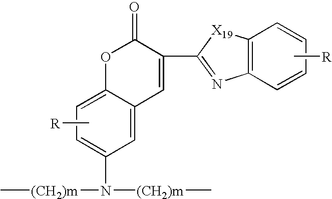

wherein: X₁₉ is a C, O, N, or S atom, and R is defined as above, and can represent more than one substituent;

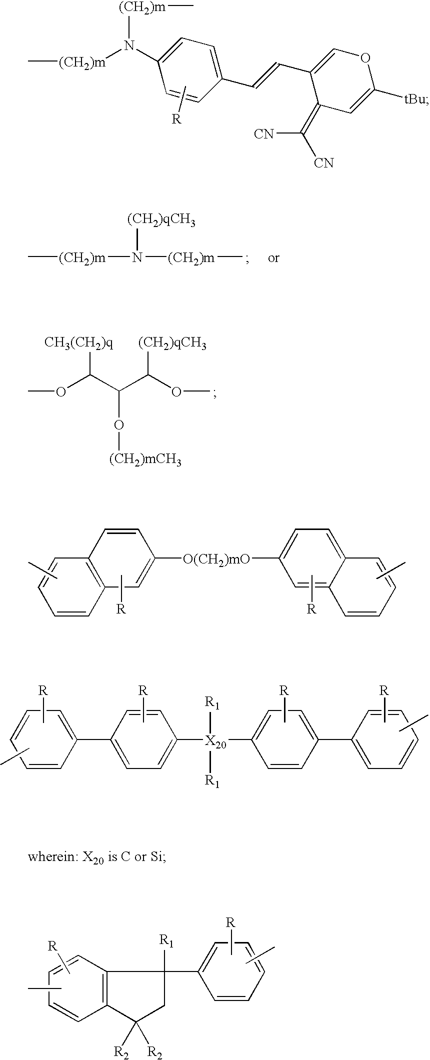

wherein: R₁ and R₂, are substituents each being individually hydrogen, or alkyl, or alkenyl, or alkynyl, or alkoxy of from 1 to 40 carbon atoms; aryl or substituted aryl of from 6 to 60 carbon atoms; or heteroaryl or substituted heteroaryl of from 4 to 60 carbons; or F, Cl, or Br; or a cyano group; or a nitro group;

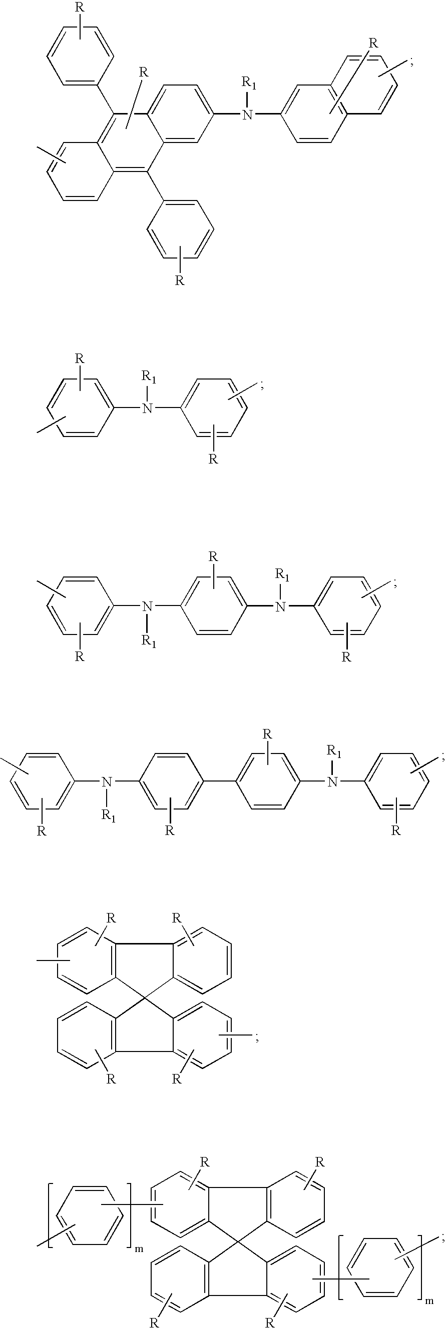

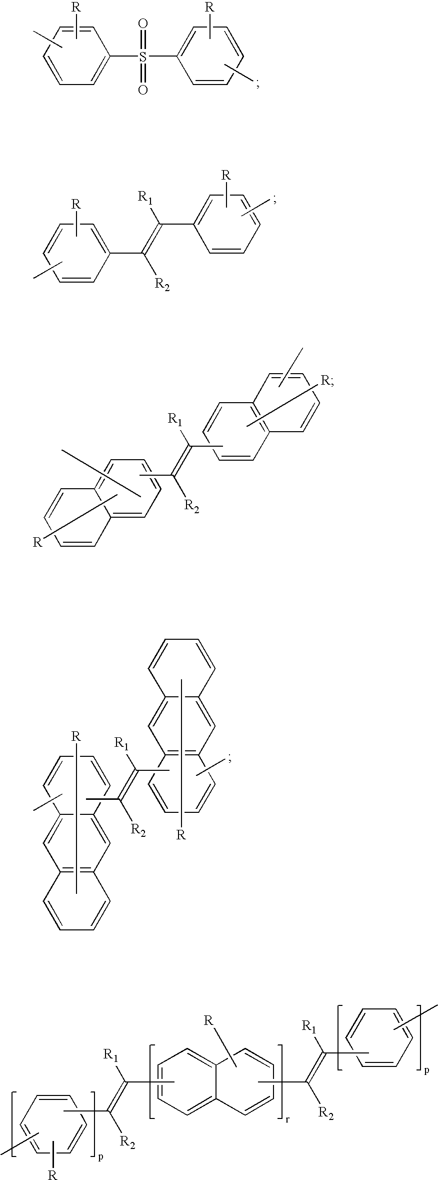

wherein: p and r are integers from 1 to 4;

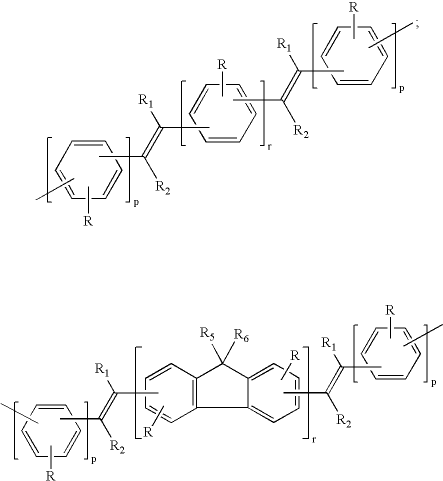

wherein: R₅, and R₆ are substituents each being individually hydrogen, or alkyl, or alkenyl, or alkynyl, or alkoxy of from 1 to 40 carbon atoms; aryl or substituted aryl of from 6 to 60 carbon atoms; or heteroaryl or substituted heteroaryl of from 4 to 60 carbons; or F, Cl, or Br; or a cyano group; or a nitro group; or

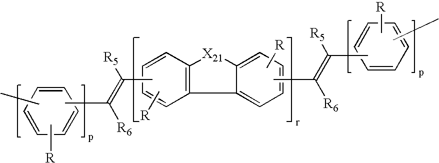

wherein: X₂₁ is O or S atom, or N—R, or R—Si—R;

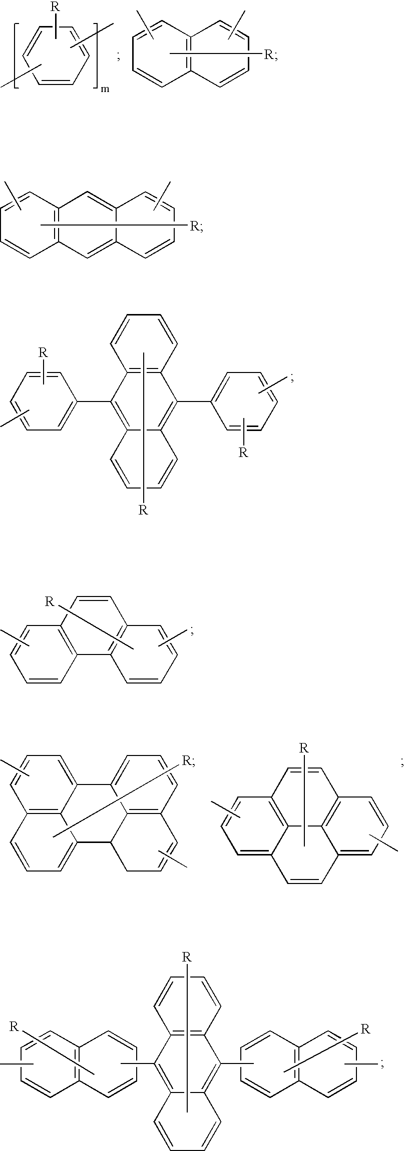

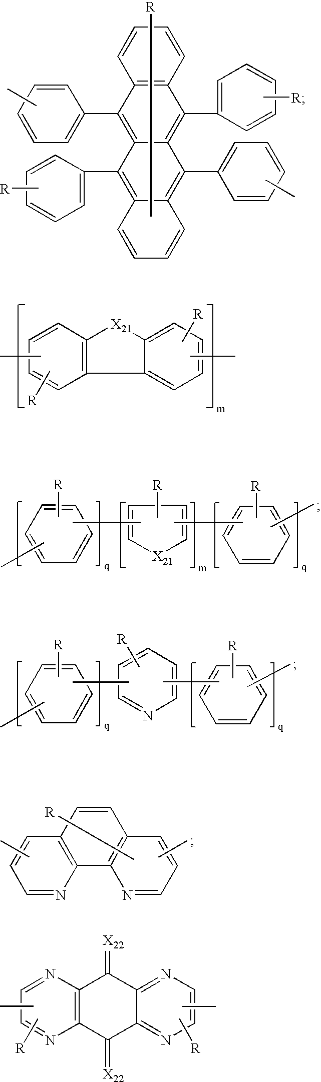

wherein: X₂₂ is an O atom or two cyano groups;

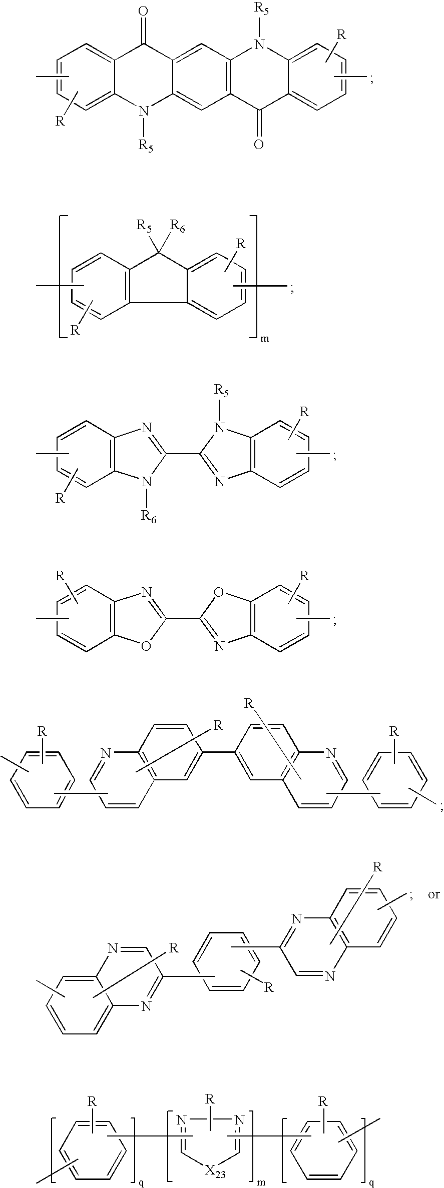

wherein: X₂₃ is O or S atom, or N—R.

The following structures constitute specific examples the 6-member ring structure of formula (I), wherein X is a carbon atom, or a nitrogen atom, or a sulfur atom, or a silicon atom, or an oxygen atom, or a phosphorus atom, or a selenium atom, or a germanium atom; and R is hydrogen, alkyl, alkynyl, alkoxy, or alkenyl group containing 1 to 40 carbon atoms; aryl or substituted aryl containing 6 to 60 carbon atoms; or heteroaryl or substituted heteroaryl containing 4 to 60 carbons; or F, Cl, or Br; or a cyano, or a nitro group. Preferably, X is a carbon atom, a nitrogen atom, a sulfur atom, a silicon atom, or an oxygen atom. More preferably, X is a carbon atom, a nitrogen atom, an oxygen atom, or a sulfur atom. Most preferably, X is a carbon atom, a nitrogen atom, or an oxygen atom.

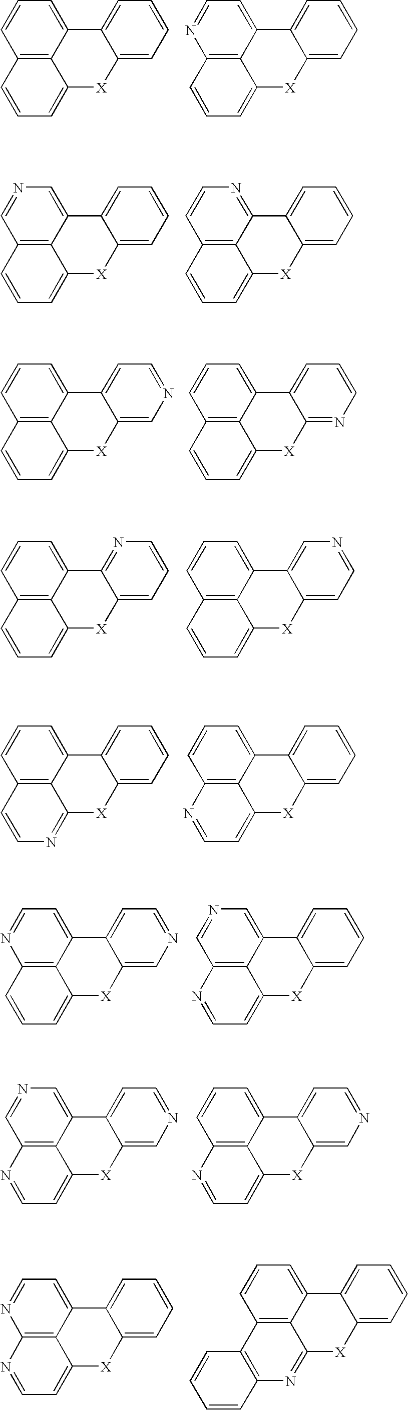

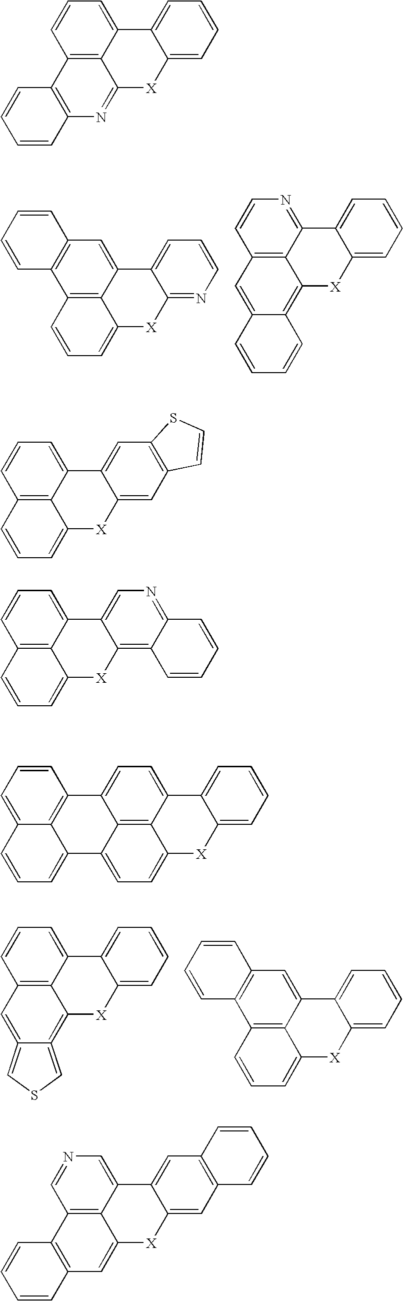

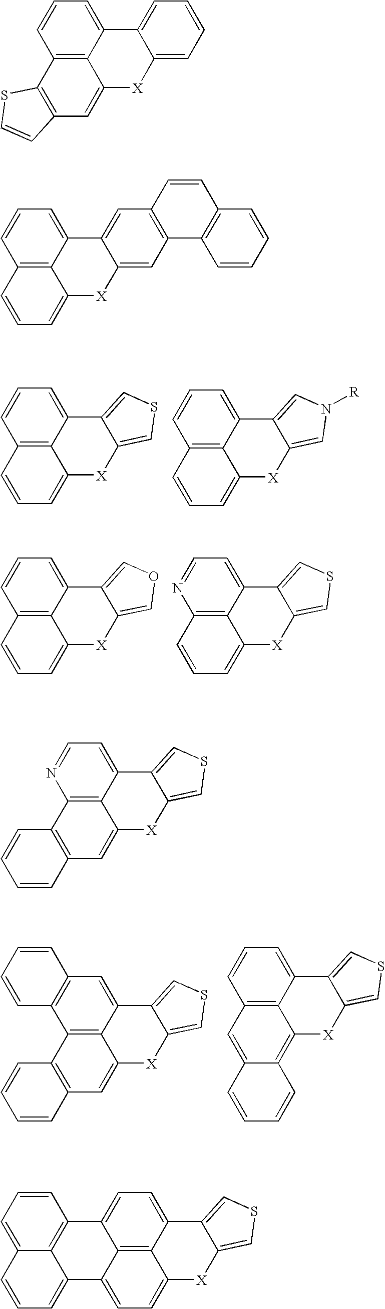

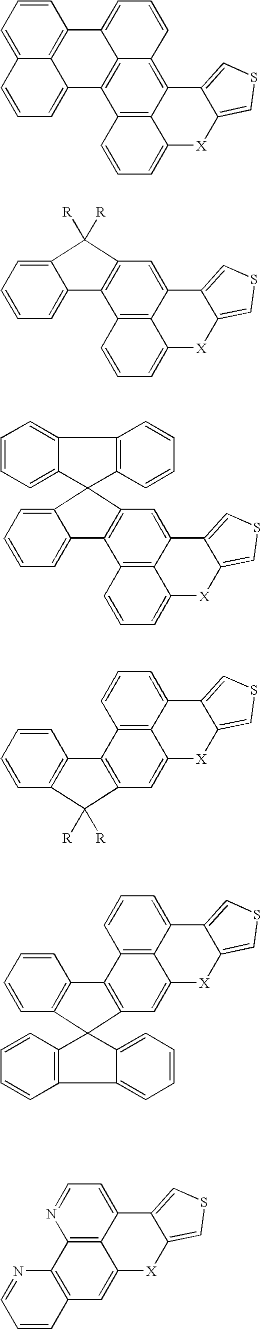

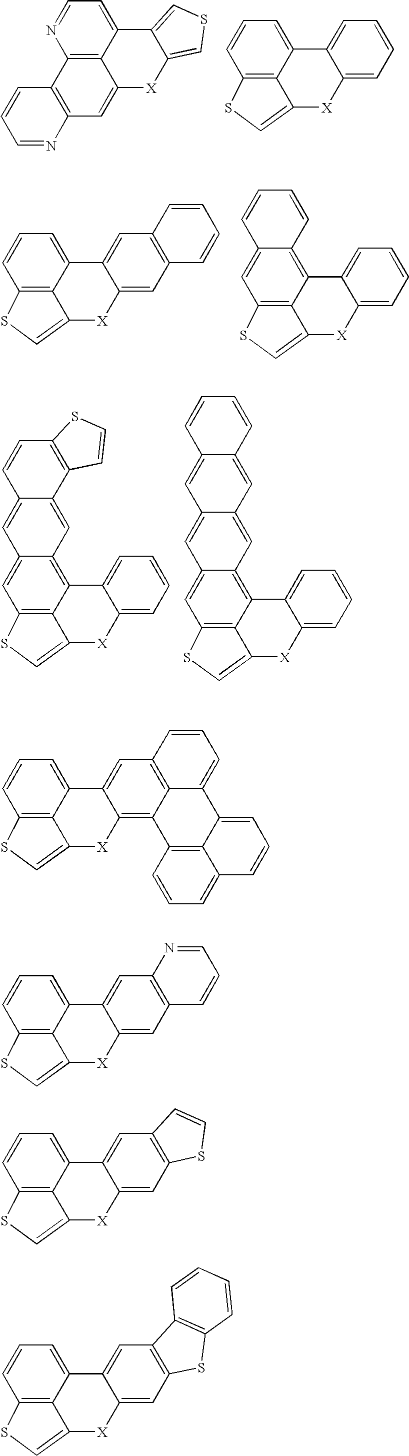

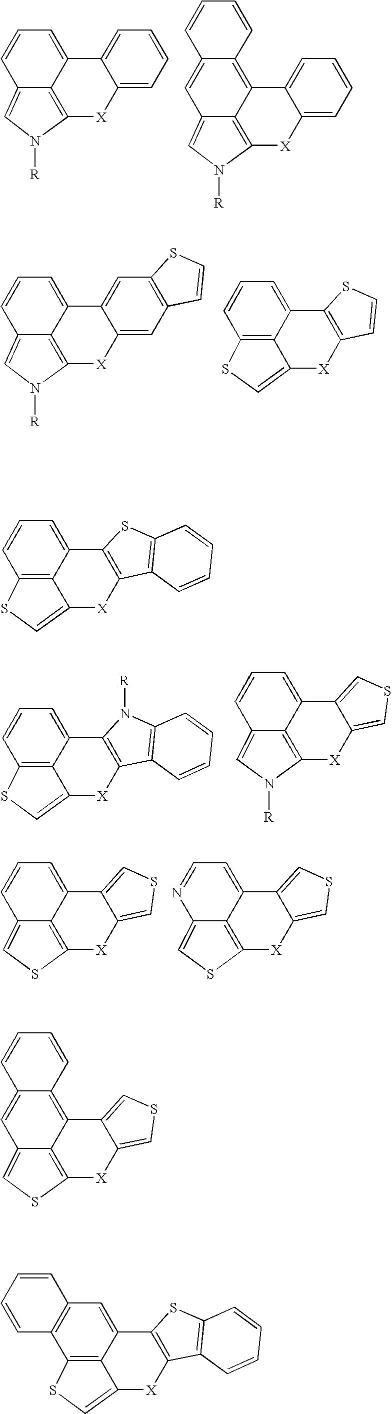

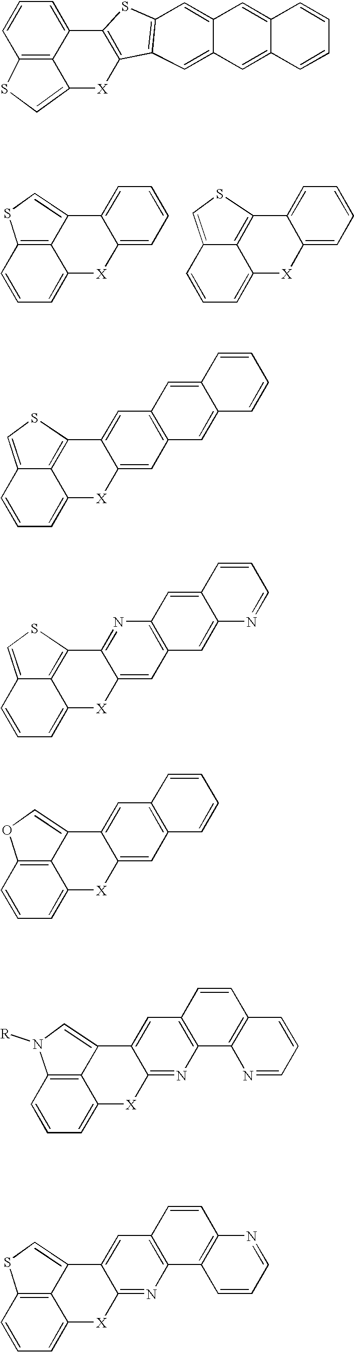

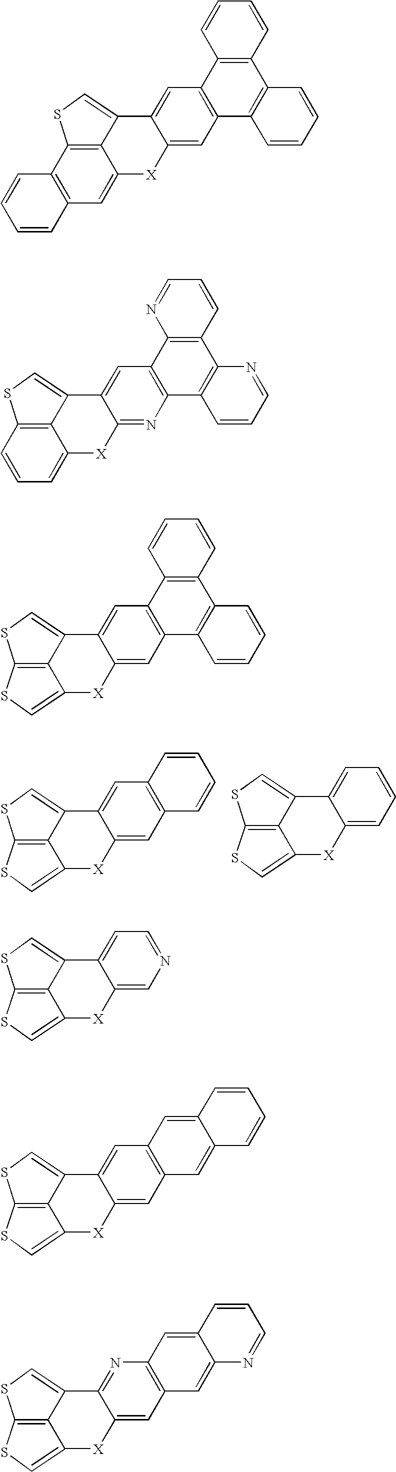

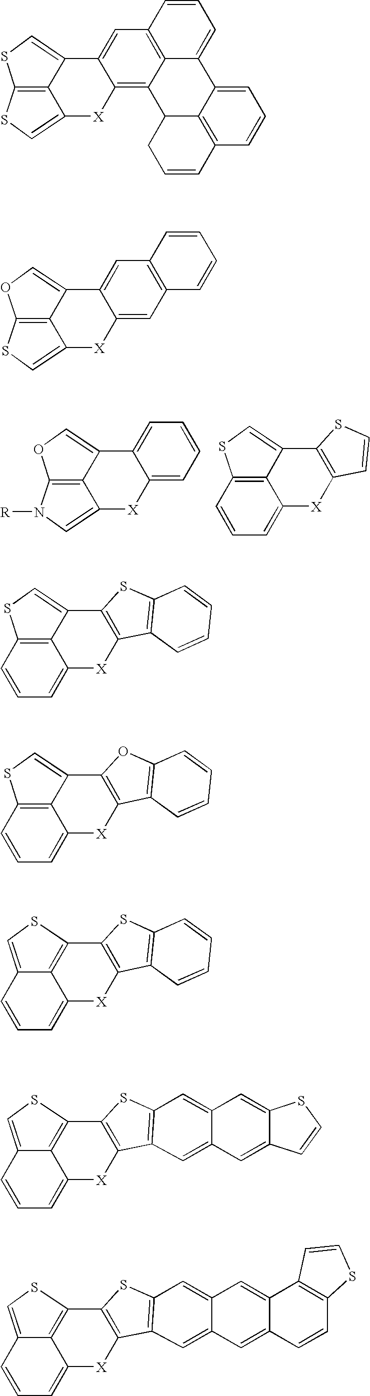

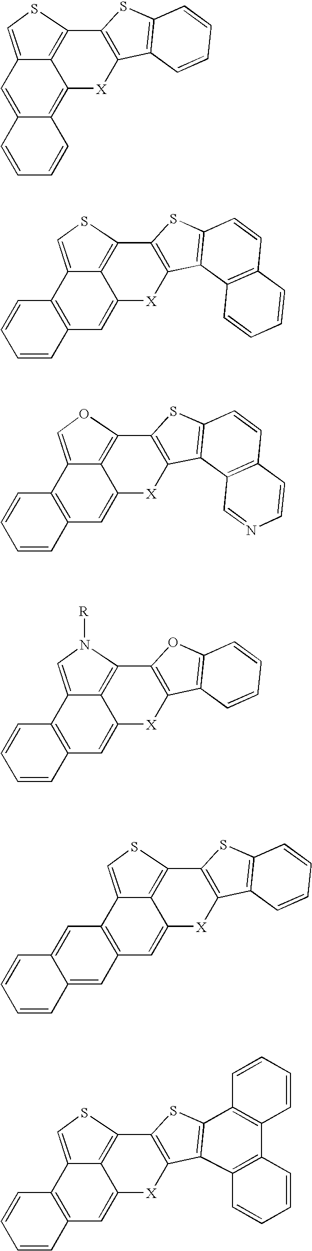

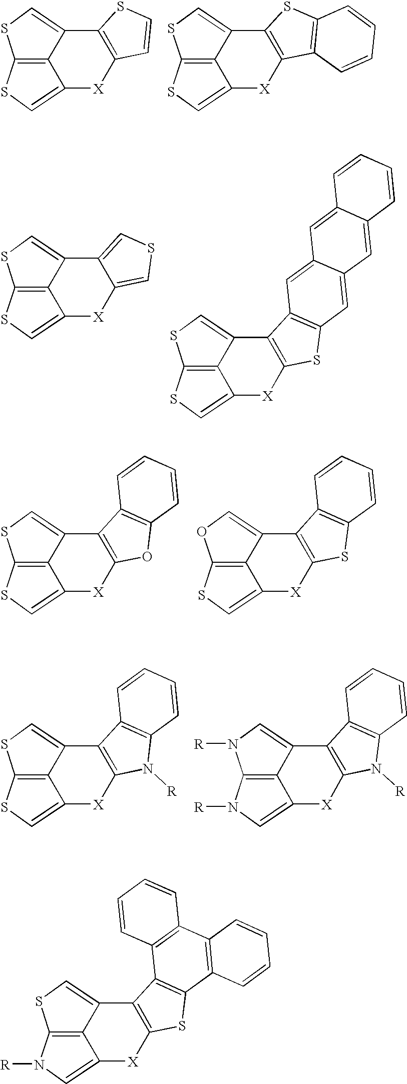

The following molecular structures constitute specific examples of preferred compounds satisfying the requirement of this invention:

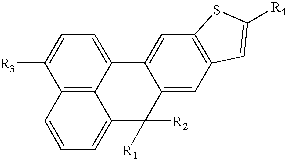

compound 1 R₁═R₂=R₃═R₄=phenyl
compound 2 R₁═R₂=hexyl, R₃═R₄=phenyl
compound 3 R₁═R₂=phenyl, R₃═R₄=2-naphthyl

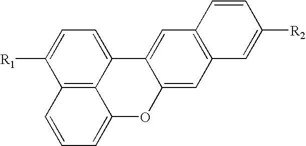

compound 4 R₁═R₂=phenyl
compound 5 R₁═R₂=2-naphthyl
compound 6 R₁=phenyl, R₃=2-naphthyl

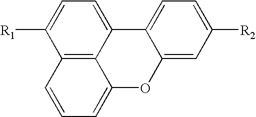

compound 7 R₁═R₂=phenyl
compound 8 R₁═R₂=2-naphthyl
compound 9 R₁=phenyl, R₃=2-naphthyl

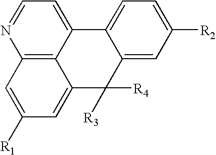

compound 10 R₁=R₂═R₃=R₄=phenyl
compound 11 R₁═R₂=hexyl, R₃═R₄=phenyl
compound 12 R₁═R₂=2-naphthyl, R₃═R₄=phenyl

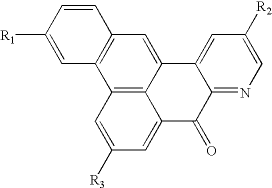

compound 13 R₁═R₂=R₃=phenyl compound 14 R₁═R₂=2-naphthyl, R₃=phenyl
compound 15 R₁=R₂═R₃=hexyl
compound 16 R₁=phenyl, R₂=2-pyridyl, R₃=hexyl

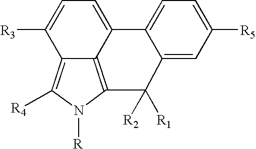

compound 17 R₁═R₂=R₃═R₄=R₅=phenyl
compound 18 R₁═R₂=hexyl, R₃═R₄=R₅=phenyl
compound 19 R₁═R₂=hexyl, R₃═R₅=2-naphthyl, R₄=phenyl

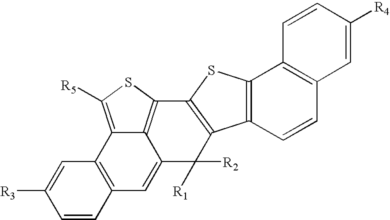

compound 20 R₁═R₂=R₃═R₄=R₅=phenyl
compound 21 R₁═R₂=hexyl, R₃═R₄=R₅=phenyl
compound 22 R₁═R₂=R₅=hexyl, R₃=2-naphthyl, R₄=phenyl

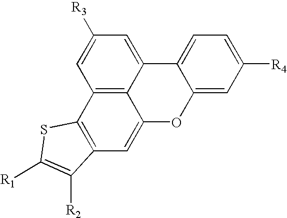

compound 23 R₁═R₂=R₃═R₄=phenyl
compound 24 R₁═R₂=hexyl, R₃═R₄=phenyl
compound 25 R₁═R₂=2-ethylhexyl, R₃═R₄=2-naphthyl

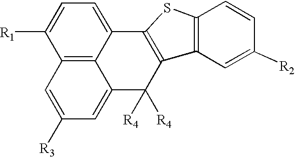

compound 26 R₁═R₂=R₃=phenyl, R₄=hexyl
compound 27 R₁═R₂=R₄=hexyl, R₃=phenyl
compound 28 R₁═R₂=R₃=2-naphthyl, R₄=2-ethylhexyl
compound 29 R₁═R₂=R₃=diphenylamino, R₄=methyl

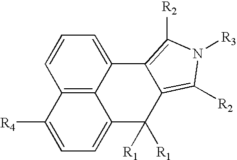

compound 30 R₁═R₃=hexyl, R₂═R₄=phenyl
compound 31 R₁═R₂=R₄=hexyl, R₃=phenyl
compound 32 R₁=2-ethylhexyl R₂═R₃=phenyl, R₄=2-naphthyl

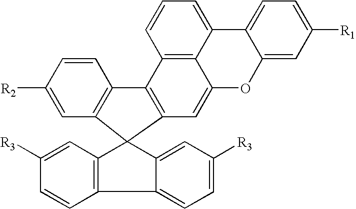

compound 33 R₁═R₃=hexyl, R₂=diphenylamino
compound 34 R₁═R₂=R₃=phenyl
compound 35 R₁═R₃=2-ethylhexyl, R₂=2-naphthyl

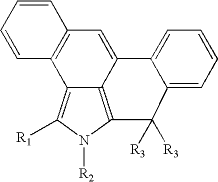

compound 36 R₁═R₂=R₃=hexyl
compound 37 R₁═R₃=phenyl, R₂=hexyl,
compound 38 R₁=2-naphthyl, R₂═R₃=2-ethylhexyl

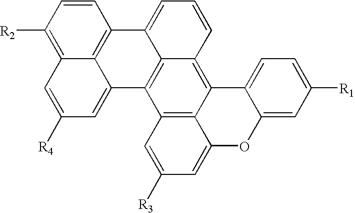

compound 39 R₁═R₃=hexyl, R₂═R₄=phenyl
compound 40 R₁═R₄=2-naphthyl, R₂═R₃=phenyl
compound 41 R₁=2-ethylhexyl, R₂═R₃=phenyl, R₄=H

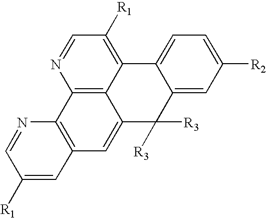

compound 42 R₁═R₂=R₃=hexyl
compound 43 R₁═R₂=R₃=phenyl
compound 44 R₁═R₂=phenyl, R₃=hexyl,
compound 45 R₁═R₂=4-pyridyl, R₃=phenyl

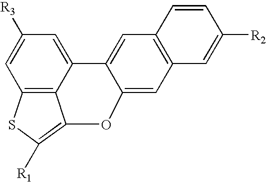

compound 46 R₁═R₂=R₃=hexyl
compound 47 R₁═R₂=R₃=phenyl
compound 49 R₁═R₂=phenyl, R₃=2-naphthyl
compound 50 R₁═R₃=phenyl, R₂=4-pyridyl

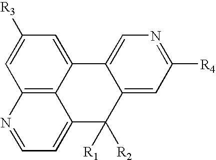

compound 51 R₁═R₂=hexyl, R₃═R₄=2-naphthyl
compound 52 R₁═R₂=methyl, R₃=H, R₄=2-naphthyl

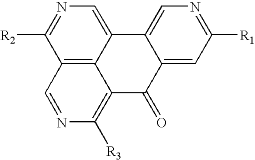

compound 53 R₁═R₂=R₃=phenyl
compound 54 R₁═R₃=phenyl, R₂=4-pyridyl
compound 55 R₁=2-naphthyl, R₂=H, R₃=phenyl

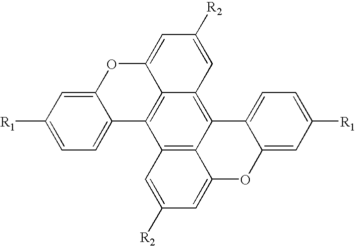

compound 56 R₁═R₂=phenyl
compound 57 R₁=hexyl, R₂=phenyl
compound 58 R₁=2-ethylhexyl, R₂=2-naphthyl

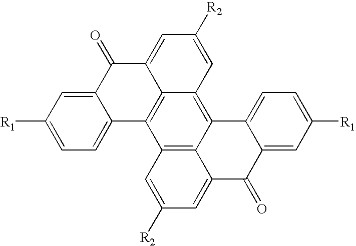

compound 59 R₁═R₂=phenyl
compound 60 R₁=hexyl, R₂=2-pyridyl

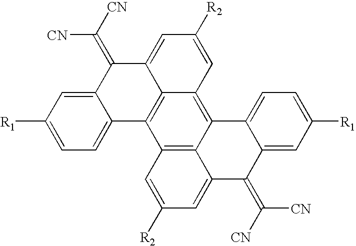

compound 61 R₁=R₂=phenyl
compound 62 R₁═R₂=hexyl
compound 63 R₁═R₂=H
compound 64 R₁═R₂=2-pyridyl

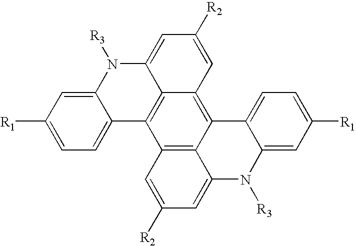

compound 65 R₁═R₂=R₃=phenyl
compound 66 R₁=diphenylamino, R₂=hexyl, R₃=phenyl
compound 67 R₁═R₂=phenyl, R₃=hexyl

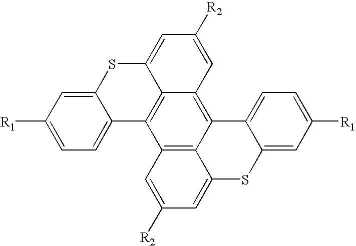

compound 68 R₁═R₂=phenyl
compound 69 R₁=hexyl, R₂=phenyl
compound 70 R₁=2-ethylhexyl, R₂=2-naphthyl

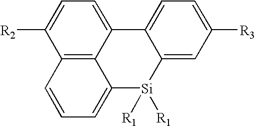

compound 71 R₁═R₂=R₃=phenyl
compound 72 R₁═R₃=phenyl, R₂=2-naphthyl
compound 73 R₁=methyl, R₂═R₃=2-naphthyl

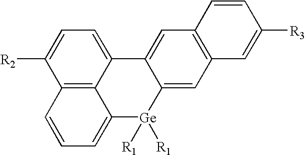

compound 74 R₁═R₂=R₃=phenyl
compound 75 R₁═R₃=phenyl, R₂=2-naphthyl
compound 76 R₁=methyl, R₂═R₃=2-naphthyl

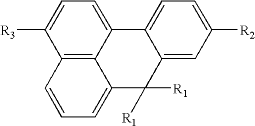

compound 77 R₁═R₂=R₃=phenyl
compound 78 R₁=H, R₂=2-naphthyl, R₃=phenyl
compound 79 R₁=methyl, R₂═R₃=2-naphthyl

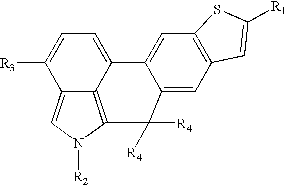

compound 80 R₁═R₂=R₃=phenyl, R₄=hexyl
compound 81 R₁═R₂=R₄=hexyl, R₃=phenyl
compound 82 R₁=2-ethylhexyl, R₂═R₄=methyl, R₃=2-naphthyl

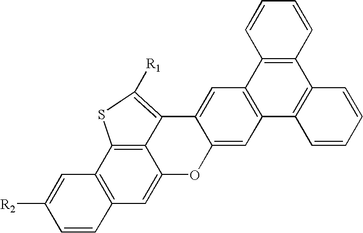

compound 83 R₁═R₂=phenyl
compound 84 R₁=hexyl, R₂=phenyl

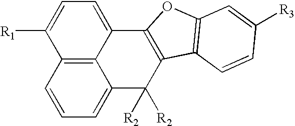

compound 85 R₁═R₂=R₃=phenyl
compound 86 R₁═R₂=R₃=hexyl
compound 87 R₁=2-ethylhexyl, R₂=methyl, R₃=2-naphthyl

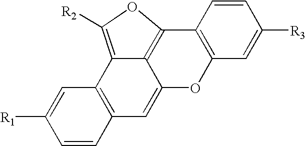

compound 88 R₁═R₂=R₃=phenyl
compound 89 R₁═R₂=R₃=hexyl
compound 90 R₁═R₃=2-naphthyl, R₂=methyl

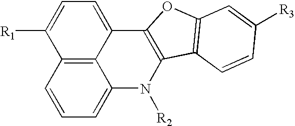

compound 91 R₁═R₂=R₃=phenyl
compound 92 R₁═R₂=R₃=hexyl
compound 93 R₁═R₃=2-naphthyl, R₂=methyl

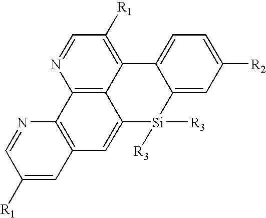

compound 94 R₁═R₂=R₃=phenyl
compound 95 R₁═R₂=R₃=hexyl
compound 96 R₁═R₂=2-naphthyl, R₃=methyl

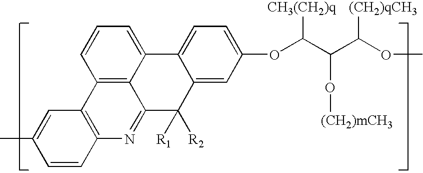

compound 97 R₁═R₂=2-ethylhexyl, m=10, q=6
compound 98 R₁═R₂=4-decylphenyl, m=2, q=5
compound 99 R₁═R₂=n-decyl, m=q=1

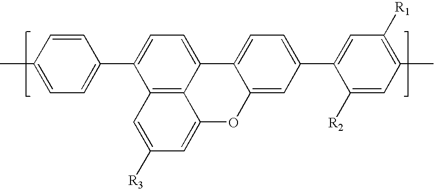

compound 100 R₁═R₂=2-ethylhexyl, R₃=2-ethylhexyl
compound 101 R₁═R₂=hexyloxy, R₃=2-ethylhexyl
compound 102 R₁═R₂=hexyloxy, R₃=H
compound 103 R₁═R₂=hexyloxy, R₃=phenyl
compound 104 R₁═R₂=diphenylamino, R₃=hexyl

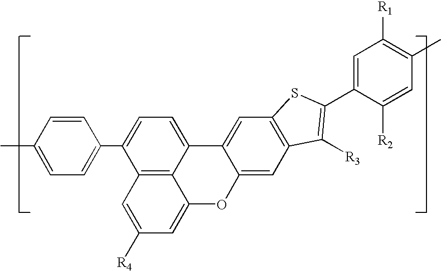

compound 105 R₁═R₂=2-ethylhexyl, R₃=2-ethylhexyl, R₄=H
compound 106 R₁═R₂=hexyloxy, R₃═R₄=2-ethylhexyl
compound 107 R₁═R₂=hexyloxy, R₃═R₄=H
compound 108 R₁═R₂=hexyloxy, R₃=phenyl, R₄=H
compound 109 R₁═R₂=diphenylamino, R₃=hexyl, R₄=phenyl

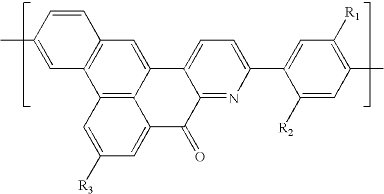

compound 110 R₁═R₂=R₃=2-ethylhexyl
compound 111 R₁═R₂═R₃=hexyloxy
compound 112 R₁═R₂=2-ethylhexyl, R₃=H

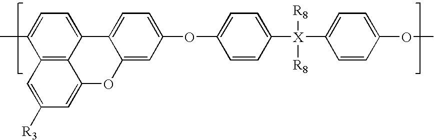

compound 113 R₃=n-hexyloxy, R₈=ethyl, X=C
compound 114 R₃=2-ethylhexyl, R₈═CF₃, X=C
compound 115 R₃=4-decylphenyl, R₈=n-butyl, X=Si
compound 116 R₃=4-(bis(4-methylphenyl)amino)phenyl, R₈=n-hexyl, X=Si

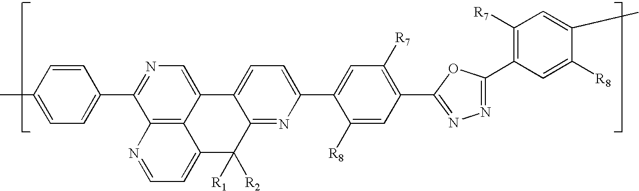

compound 117 R₁═R₂=n-hexyl, R₇═R₈=2-ethylhexyloxy
compound 118 R₁═R₂═R₇=3,7-dimethyloctyl, R₈=phenyl
compound 119 R₁=hexyl, R₂=n-decyl, R₇=t-butyl, R₈=n-hexyloxy

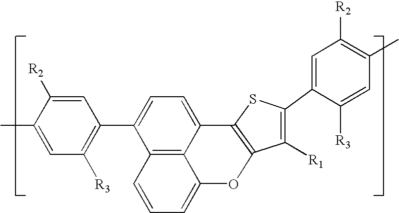

compound 120 R₁═R₂=R₃=n-hexyl
compound 121 R₁═R₂=R₃=3,7-dimethyloctyloxy
compound 122 R₁═R₃=hexyl, R₂=hexyloxy

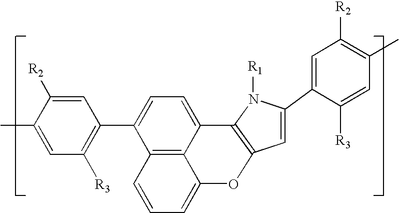

compound 123 R₁═R₂=R₃=n-hexyl
compound 124 R₁=methyl, R₂═R₃=3,7-dimethyloctyloxy
compound 125 R₁═R₃=hexyl, R₂=hexyloxy
compound 126 R₁=phenyl, R₃═R₂=hexyloxy

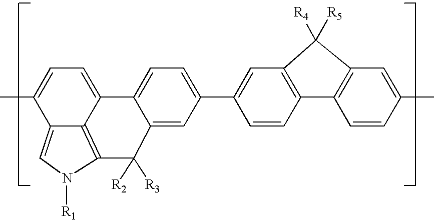

compound 127 R₁═R₂=R₃=n-hexyl
compound 128 R₁=phenyl, R₂═R₃=R₄═R₅=3,7-dimethyloctyl
compound 129 R₁=phenyl, R₂═R₃=ethyl, R₄═R₅=hexyl

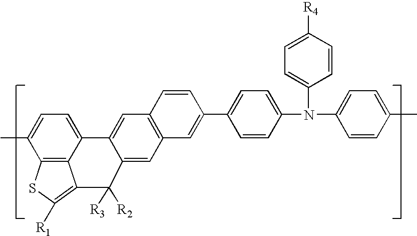

compound 130 R₁═R₂=R₃═R₄=n-hexyl
compound 131 R₁=hexyl, R₂═R₃=octyl, R₄=diphenylamino
compound 132 R₁=phenyl, R₂═R₃=ethyl, R₄=t-butyl

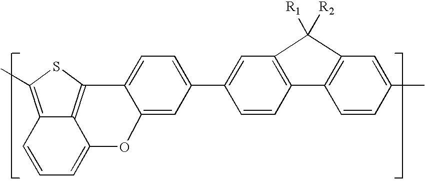

compound 133 R₁═R₂=n-hexyl
compound 134 R₁=hexyl, R₂=octyl
compound 135 R₁═R₂=4-hexylphenyl

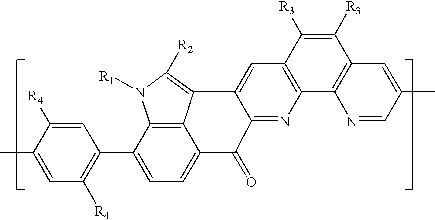

compound 136 R₁═R₂=R₃═R₄=n-hexyl
compound 137 R₁=hexyl, R₂═R₃=octyl, R₄=diphenylamino
compound 138 R₁═R₃=phenyl, R₂=H, R₄=hexyloxy

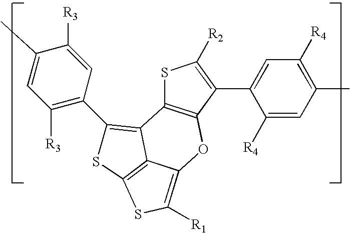

compound 139 R₁═R₂=R₃═R₄=n-hexyl
compound 140 R₁═R₂=hexyl, R₃═R₄=octyloxy
compound 141 R₁=phenyl, R₃=H, R₂=hexyl, R₄=hexyloxy

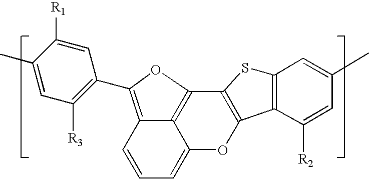

compound 142 R₁═R₂=R₃=octyloxy
compound 143 R₁═R₂=R₃=hexyl
compound 144 R₁=methoxy, R₂=H, R₃=3,7-dimethyloctyloxy

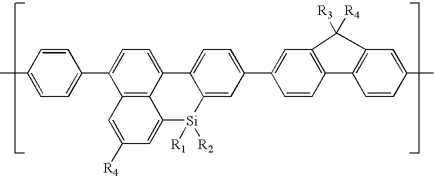

compound 145 R₁═R₂=R₃═R₄=n-hexyl
compound 146 R₁═R₂=methyl, R₃═R₄=octyl
compound 147 R₁═R₂=phenyl, R₃═R₄=octyl

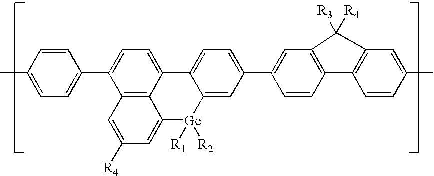

compound 148 R₁═R₂=R₃═R₄=n-hexyl
compound 149 R₁═R₂=methyl, R₃═R₄=octyl
compound 150 R₁═R₂=phenyl, R₃═R₄=octyl
compound 151 R₁═R₂=R₃═R₄=4-tert-butylphenyl

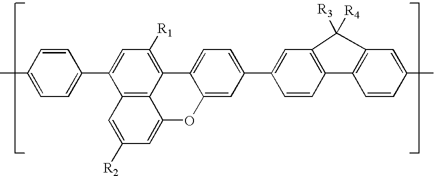

compound 152 R₁═R₂=R₃═R₄=n-hexyl
compound 153 R₁═R₂=H, R₃═R₄=octyl
compound 154 R₁═R₂=4-hexylphenyl, R₃═R₄=octyl
compound 155 R₁═R₂=hexyl, R₃═R₄=4-tert-butylphenyl

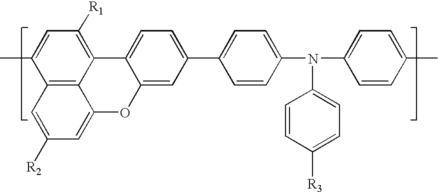

compound 156 R₁═R₂=R₃=n-hexyl
compound 157 R₁═R₂=hexyloxy, R₃=t-butyl
compound 158 R₁=4-hexylphenyl, R₂═R₃=H
compound 159 R₁═R₂=octyl, R₃=diphenylamino

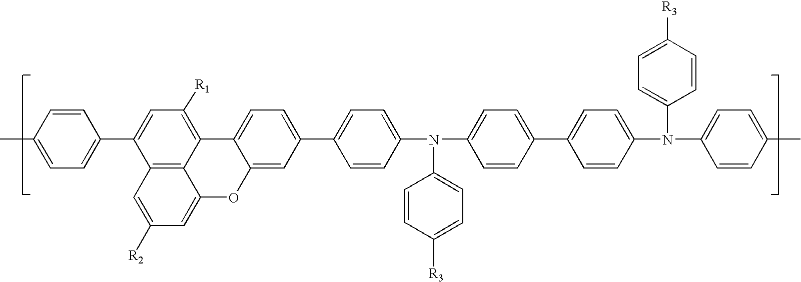

compound 160 R₁═R₂=R₃=n-hexyl
compound 161 R₁═R₂=hexyloxy, R₃=t-butyl
compound 162 R₁=methoxy, R₂=octyloxy, R₃=diphenylamino

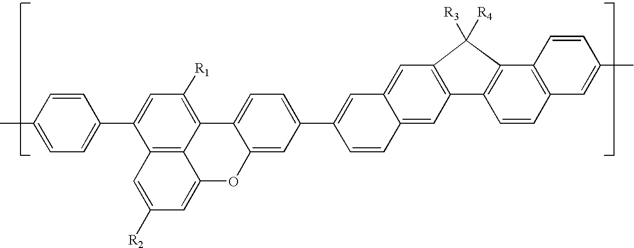

compound 163 R₁═R₂=R₃═R₄=n-hexyl
compound 164 R₁=R₂=H, R₃═R₄=octyl
compound 165 R₁═R₂=4-hexylphenyl, R₃═R₄=octyl
compound 166 R₁═R₂=hexyl, R₃═R₄=4-tert-butylphenyl

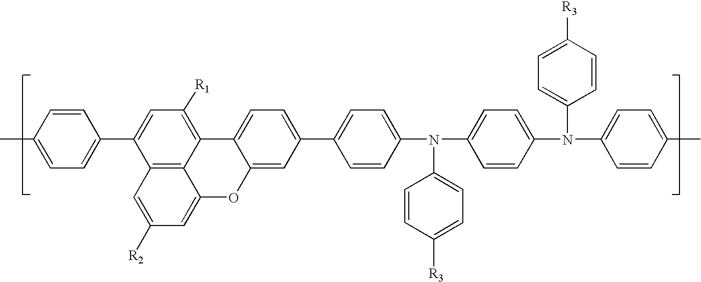

compound 167 R₁═R₂=R₃=n-hexyl
compound 168 R₁═R₂=hexyloxy, R₃=t-butyl
compound 169 R₁=methoxy, R₂=octyloxy, R₃=diphenylamino

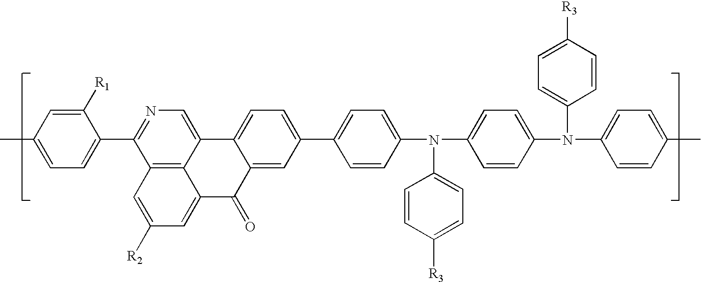

compound 170 R₁═R₂=R₃=n-hexyl
compound 171 R₁═R₂=hexyloxy, R₃=t-butyl
compound 172 R₁=methoxy, R₂=octyloxy, R₃=COOH

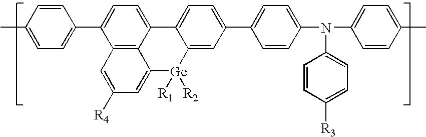

compound 173 R₁═R₂=R₃=n-hexyl
compound 174 R₁═R₂=hexyl, R₃═R₄=hexyloxy
compound 175 R₁═R₂=4-hexylphenyl, R₃═R₄=t-butyl
compound 176 R₁═R₂=octyl, R₃═R₄=diphenylamino

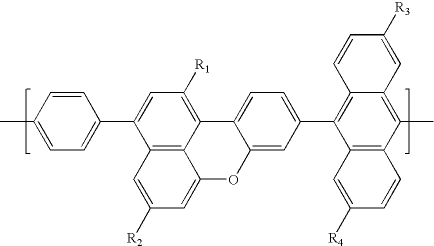

compound 177 R₁═R₂=R₃=n-hexyl
compound 178 R₁═R₂=hexyl, R₃═R₄=hexyloxy
compound 179 R₁═R₂=H, R₃═R₄=octyl
compound 180 R₁═R₂=octyl, R₃═R₄=diphenylamino

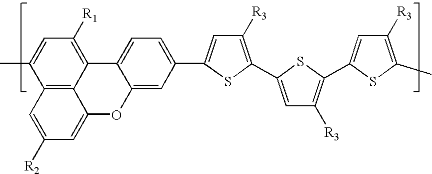

compound 181 R₁═R₂=R₃=n-hexyl
compound 182 R₁═R₂=hexyloxy, R₃=t-butyl
compound 183 R₁=methoxy, R₂=octyloxy, R₃=diphenylamino

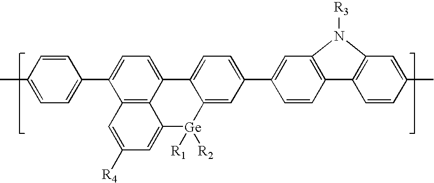

compound 184 R₁═R₂=R₃═R₄=n-hexyl
compound 185 R₁═R₂=R₃=hexyl, R₄=hexyloxy
compound 186 R₁═R₂=R₃=4-hexylphenyl, R₄=H
compound 187 R₁═R₂=octyl, R₃═R₄=diphenylamino

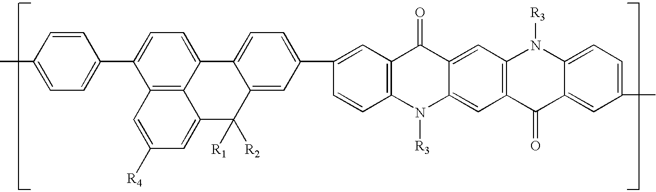

compound 188 R₁═R₂=R₃═R₄=n-hexyl
compound 189 R₁═R₂=R₃=hexyl, R₄=hexyloxy
compound 190 R₁═R₂=R₃=4-hexylphenyl, R₄=H

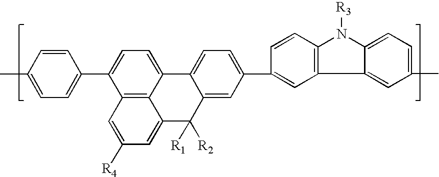

compound 191 R₁═R₂=R₃=hexyl, R₄=hexyloxy
compound 192 R₁═R₂=R₃=4-hexylphenyl, R₄=H
compound 193 R₁═R₂=octyl, R₃═R₄=diphenylamino

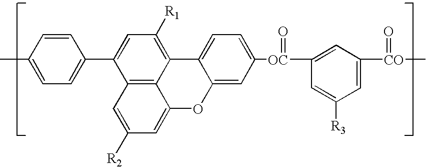

compound 194 R₁═R₂=R₃=n-hexyl
compound 195 R₁═R₂=hexyloxy, R₃=COOH
compound 196 R₁=methoxy, R₂=octyloxy, R₃═SO₃H

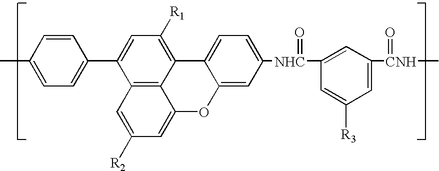

compound 197 R₁═R₂=R₃=n-hexyl
compound 198 R₁═R₂=hexyloxy, R₃=COOH
compound 199 R₁=methoxy, R₂=octyloxy, R₃═SO₃H

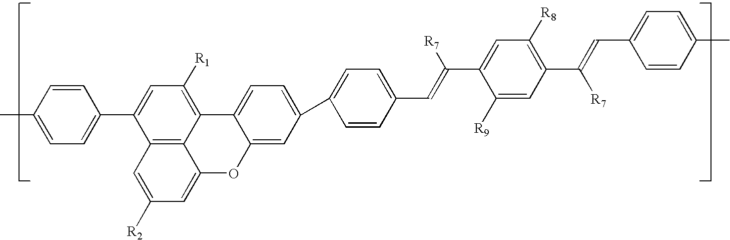

compound 200 R₁═R₂=n-hexyl, R₇=phenyl, R₈═R₉=2-ethylhexyl
compound 201 R₁═R₂=n-hexyl, R₇=H, R₈=methoxy, R₉=3,7-dimethyloctyloxy
compound 202 R₁=n-hexyl, R₂═R₈=3,7-dimethyloctyl, R₇=CN, R₉=(4-diphenylamino)phenyl

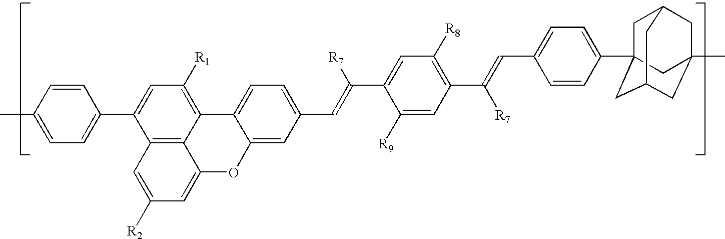

compound 203 R₁═R₂=n-hexyl, R₇=phenyl, R₈═R₉=2-ethylhexyl
compound 204 R₁═R₂=n-hexyl, R₇=H, R₈=methoxy, R₉=3,7-dimethyloctyloxy
compound 205 R₁=n-hexyl, R₂═R₈=3,7-dimethyloctyl, R₇=CN, R₉=(4-diphenylamino)phenyl

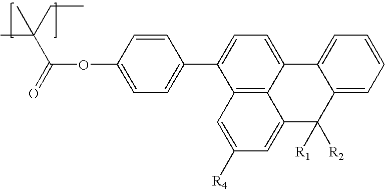

compound 206 R₁═R₂=hexyl, R₄=hexyloxy
compound 207 R₁═R₂=4-hexylphenyl, R₄=H
compound 208 R₁═R₂=octyl, R₄=diphenylamino

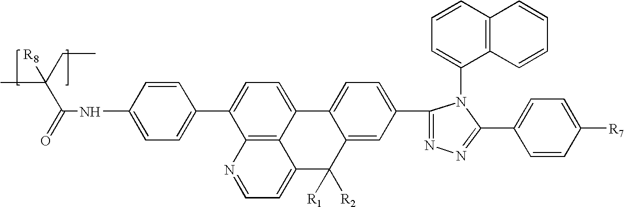

compound 209 R₁=2-ethylhexyl, R₂=n-hexyl, R₇=t-butyl, R₈=H
compound 210 R₁═R₂=n-hexyl, R₇=phenyl, R₈═CN
compound 211 R₁=4-decylphenyl, R₂═R₇=3,7-dimethyloctyl, R₈=methyl

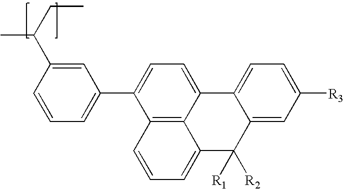

compound 212 R₁═R₂=R₃=n-hexyl
compound 213 R₁═R₂=hexyl, R₃=hexyloxy
compound 214 R₁═R₂=4-hexylphenyl, R₃=diphenylamino

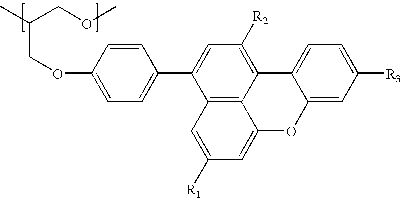

compound 215 R₁═R₂=R₃=n-hexyl
compound 216 R₁═R₂=hexyl, R₃=hexyloxy
compound 217 R₁=H, R₂=4-hexylphenyl, R₃=diphenylamino

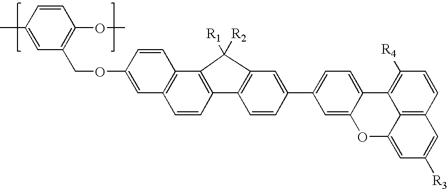

compound 218 R₁═R₂=R₃═R₄=n-hexyl
compound 218 R₁═R₂=hexyl, R₃═R₄=hexyloxy
compound 219 R₁═R₂=R₃=4-hexylphenyl, R₄=H

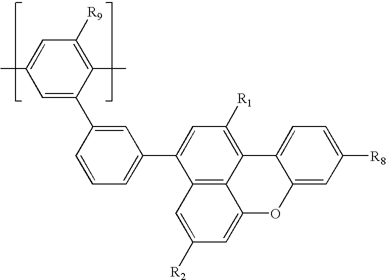

compound 220 R₁═R₂=2-ethylhexyloxy, R₈=4-t-butylphenyl, R₉=H
compound 221 R₁═R₂=n-hexyl, R₈═R₉=n-hexyloxy
compound 222 R₁═R₂=4-decylphenyl, R₉=CN, R₈=(4-diphenylamino)phenyl

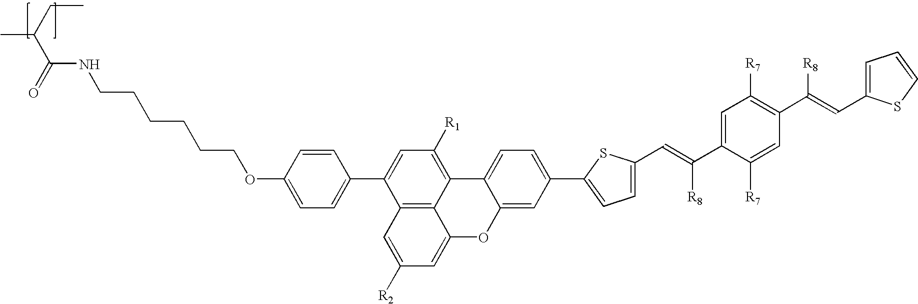

compound 223 R₁=2-ethylhexyl, R₂=n-hexyl, R₇=t-butyl, R₈=H
compound 224 R₁═R₂=n-hexyl, R₇=n-octyloxy, R₈=CN
compound 225 R₁=4-decylphenyl, R₂═R₇=3,7-dimethyloctyl, R₈=CN

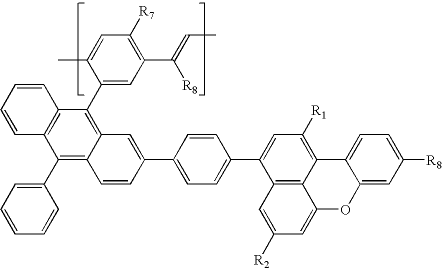

compound 226 R₁=2-ethylhexyl, R₂=n-hexyl, R₇=n-hexyl, R₈=H
compound 227 R₁═R₂=n-hexyl, R₇=n-octyloxy, R₈=CN
compound 228 R₁═R₇=4-decylphenyl, R₂=3,7-dimethyloctyl, R₈=CN

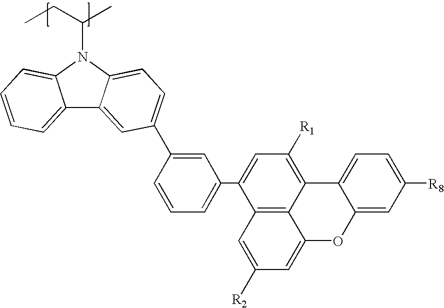

compound 229 R₁═R₂=R₈=2-ethylhexyl
compound 230 R₁=H, R₂=n-hexyl, R₈=(4-diphenylamino)phenyl
compound 231 R₁=n-hexyloxy, R₂=(4-diphenylamino)phenyl, R₈=H

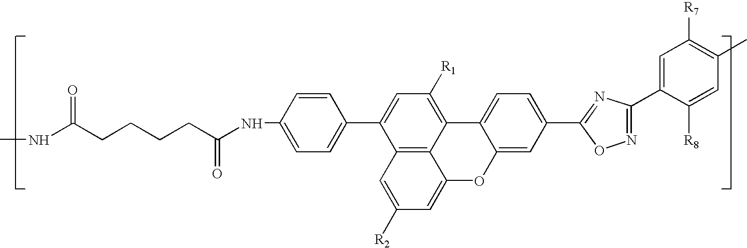

compound 232 R₁=2-ethylhexyl, R₂=n-hexyl, R₇=t-butyl, R₈=n-butyloxy
compound 233 R₁═R₂=n-hexyl, R₇=phenyl, R₈=H
compound 234 R₁=4-decylphenyl, R₂═R₇=3,7-dimethyloctyl, R₈=methoxy

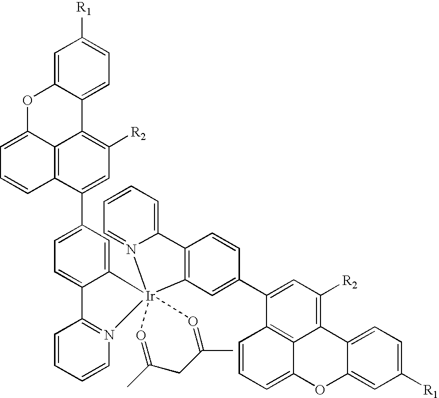

compound 235 R₁═R₂=2-ethylhexyl
compound 236 R₁═R₂=R₈=H
compound 237 R₁═R₂=phenyl

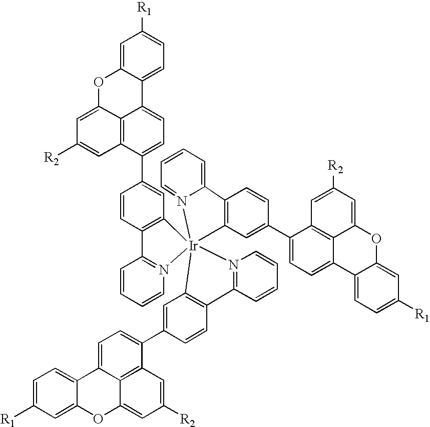

compound 238 R₁═R₂=2-ethylhexyl
compound 239 R₁═R₂=R₈=H
compound 240 R₁═R₂=phenyl

The specific molecular structures can be the combination of any of the above drawn structures.

Organic compounds comprising 6-member ring structures (Ia)-(Ih) can be synthesized using known methods. For polymers, the polymerization method and the molecular weights of the resulting polymers used in the present invention are not necessary to be particularly restricted. The molecular weights of the polymers are at least 1000, and preferably at least 2000. The polymers may be prepared by condensation polymerizations, such as coupling reactions including Pd-catalyzed Suzuki coupling, Stille coupling or Heck coupling, or N1-mediated Yamamoto coupling, or by condensation reaction between di-(acid chlorides) with di-amines, di-alcohols or di-phenols in the presence of bases, or by other condensation methods such as Wittig reaction, or Horner-Emmons reaction, or Knoevenagel reaction, or dehalogenation of dibenzyl halides, or by free radical polymerization of vinyl compounds, or ring-opening polymerization cyclic compounds, or ring-opening metathesis polymerization. Preferably polymers are prepared by Suzuki coupling reaction.

Suzuki coupling reaction was first reported by Suzuki et al on the coupling of aromatic boronic acid derivatives with aromatic halides (Suzuki, A. et al Synthetic Comm. 1981, 11(7), 513). Since then, this reaction has been widely used to prepared polymers for various applications (Ranger, M. et al Macromolecules 1997, 30, 7686). The reaction involves the use of a palladium-based catalyst such as a soluble Pd compound either in the state of Pd (II) or Pd (0), a base such as an aqueous inorganic alkaline carbonate or bicarbonate, and a solvent for the reactants and/or product. The preferred Pd catalyst is a Pd (0) complex such as Pd(PPh₃)₄ or a Pd (II) salt such as Pd(PPh₃)₂Cl₂ or Pd(OAc)₂ with a tertiary phosphine ligand, and used in the range of 0.01-10 mol % based on the functional groups of the reactants. Polar solvents such as THF and non-polar solvents toluene can be used however, the non-polar solvent is believed to slow down the reaction. Modified processes were reported to prepare conjugated polymers for EL devices from the Suzuki coupling of aromatic halides and aromatic boron derivatives (Inbasekaran, M. et al U.S. Pat. No. 5,777,070 (1998); Towns, C. R. et al. PCT WO00/53656, 2000). A variation of the Suzuki coupling reaction replaces the aromatic halide with an aromatic trifluoromethanesulfonate (triflate) (Ritter, K. Synthesis, 1993, 735). Aromatic triflates are readily prepared from the corresponding phenol derivatives. The advantages of using aromatic triflates are that the phenol derivatives are easily accessible and can be protected/deprotected during complex synthesis. For example, aromatic halides normally would react under various coupling conditions to generate unwanted by-product and lead to much more complicated synthetic schemes. However, phenol derivatives can be easily protected by various protecting groups which would not interfere with functional group transformation and be deprotected to generate back the phenol group which then can be converted to triflates. The diboron derivatives can be prepared from the corresponding dihalide or ditriflate.

The present invention also provides a process for preparing a conjugated polymer which comprises in the polymerization reaction mixture (a) an aromatic monomer having at least two reactive triflate groups and an aromatic monomer having at least two reactive boron derivative groups selected from boronic acid, boronic ester, or borane groups or an aromatic monomer having one reactive triflate group and one boron derivative group selected from boronic acid, boronic ester, or borane groups, (b) a catalytic amount of a palladium catalyst, (c) an organic or inorganic base, and (d) an organic solvent. The process of the invention produces conjugated polymers with relatively low polydispersity, high molecular weight in a relatively short reaction time. The term “conjugated polymer” refers to either a fully conjugated polymer which is conjugated along the full length of its chain and processes a delocalized pi-electron system along the chain, or a partially conjugated polymer which contains both conjugated and non-conjugated segments.

The aromatic monomers used to form conjugated polymers of the present invention must have the appropriate functional groups: the triflate and boron derivative groups. The term aromatic or aryl refers to any monomer which has triflate or boron derivative groups attached directly to the aromatic or heteroaromatic rings. The present process can be used to polymerize two systems to form a linear polymer: 1) an aryl di-triflate monomer containing two reactive triflate groups and an aryl di-boron monomer containing two reactive boron derivative functional groups; and 2) an aryl monomer containing both reactive triflate and boron derivative functional groups. To prepare branched or hyperbranched polymers using the process of the invention, in a two monomers system, both aryl monomers must contain at least two reactive triflate or boron derivative groups; in a one monomer system, the monomer must contain at least one of the triflate or boron derivative groups and more than one the other group. The boron derivative functional groups are selected from a boronic acid group represented by B(OH)₂, a boronic ester group represented by B(OR₁₂)(OR₁₃) wherein R₁₂ is substituted or unsubstituted alkyl group of 1 to 6 carbons, and R₁₃ is hydrogen, or substituted and unsubstituted alkyl group of 1 to 6 carbons, R₁₂ and R₁₃ can be the same or different, and R₁₂ and R₁₃ can be connected to form a cyclic boronic ester, preferably a 5- or 6-membered ring; and a borane group represented by BR₁₄R₁₅, wherein R₁₄ and R₁₅ are each substituted and unsubstituted alkyl group of 1 to 20 carbons. The boron derivative groups are preferably boronic acid or cyclic boronic ester groups. Polymers can be prepared by using a mixture of monomers to form copolymers with desired properties and architecture. To prepare linear polymers, the polymerization system preferably comprises about equal mole percent of the reactive triflate and boron derivative groups. The mole ratio of these two classes of reactive groups is preferably 0.98 to 1.10, more preferably less than 1.05, most preferably 1.00. If desired, a mono-functional triflate or boron derivative can be used to end-cap the chain ends.

Examples of the aryl groups for the monomers include but are not limited to aromatic hydrocarbons such as phenyl, naphthyl, anthracene, fluorene, benzofluorene, dibenzofluorene, phenanthrene, perylene, pyrene, rubrene, chrysene, tetracene, pentacene, triphenylene, diphenylanthracene, dinapthylanthracene, and benzo[a]pyrene; and heteroaromatic groups such as thiophene, pyrrole, furan, pyridine, triazines, tetrazenes, oxazoles, imidazoles, oxadiazole, thiadiazole, benzoxazole, quinoline, benzimidazole, carbazole, benzothiazole, and acridine; and triarylamines such as triphenylamine, dinaphthylphenylamine, and N,N′-diphenylbenzidine. Preferably, the aryl groups are selected from fluorene, benzofluorene, diphenylanthracene, dinaphthylanthracene, thiophene, oxadiazole, benzothiazole, benzimidazole and carbazole.

The bases suitable for use in the process of the invention include inorganic aqueous bases such as alkali metal hydroxides, carbonates acetates, and bicarbonates, alkaline earth metal hydroxides, carbonates acetates, and bicarbonates, alkaline earth metal alkoxides, and alkali metal alkoxides, and organic bases such as sources of hydroxyl ions and Lewis bases such as those which create a source of hydroxyl ions in the presence of water. The organic base should be soluble in an organic solvent and/or water. Examples of aqueous inorganic bases include the hydroxide, carbonates and bicarbonates of lithium, sodium, potassium, cesium, and barium. Preferably, the aqueous base is a solution of sodium, potassium, or cesium carbonate in a concentration of 1 to 2 M. Examples of organic bases include alkyl ammonium hydroxides, carbonates, bicarbonates, fluorides, and borates, pyridines, organic amines. Preferably, the organic base used in the process of the invention is a tetraalkylammonium hydroxide, carbonate, or bicarbonate such as tetramethyl-, tetraethyl-, or tetrapropyl-ammonium hydroxide, carbonate, or bicarbonate. The amount of base used in the process is not particularly important as long as the number of moles of the base is equal or higher than that of the monomer. Preferably, 1 to 10 molar equivalents of the base per boron-derivative functional group are employed. More preferably, 1 to 5 molar equivalents of base are used. Most preferably, 1.5 to 4 molar equivalents, and in particular 1.8 to 2.5 molar equivalents of base are used. A single base or a mixture of different bases can be used in the process of the invention.

The catalyst used in the process of the invention is preferably a palladium catalyst in a form of Pd(0) or Pd(II) complexes with ligands or Pd(II) salts. Examples of the suitable ligands for the palladium complexes are phosphines such as trialkylphosphines, tricycloalkylphosphines and triarylphosphines, where the three substituents on the phosphorus can be identical or different and one or more of the ligands can link phosphorus groups of a plurality of phosphines, where part of this linkage can also be one or me metal atoms, diketones such asdibenzylideneacetone (dba), acetylacetone and octafluoroacetylacetone, and tertiary amines such as triethylamine, trimethylamine, tripropylamines. These ligands can also be derivatized by attachment of cationic or anionic groups to render water solubility. It is also possible to use a mixture of more than one ligand. Particular examples of the phosphine ligands used in the process of the invention are trimethylphosphine, tributylphosphine, tricyclohexylphosphine, tritolylphosphine, 1,2-bis(diphenylphosphino)ethane, triphenylphosphine, 1,3-bis(diphenylphosphino)propane, and 1,1′-(diphenylphosphineo)ferrocene (dppf). Preferably, the ligands are triphenylphosphine (Ph₃P), 1,1′-(diphenlphosphineo)ferrocene (dppf), 1,2-bis(diphenylphosphino)ethane, and 1,3-(bisdiphenylphosphino)propane, and more preferably, triphenylphosphine (Ph₃P), and 1,1′-(diphenlphosphineo)ferrocene (dppf). The most preferred Pd(0) complex is Ph(Ph₃P)₄. The preferred Pd(II) salts are palladium acetate, palladium (II) propionate, palladium (II) butanoate, and palladium (II) chloride, and more preferred Pd (II) salt is palladium (II) acetate. When a palladium (II) salt is used, it is advantageous to add to the reaction mixture 2 to 4 molar equivalents of other ligands such as Ph₃P or dppf per mole of Pd salt. A Pd(II) complex such as PdCl₂(PPh₃)₂, bis(acetonitrile)palladium dichloride, dichlorobis(dimethylsulfoxide) palladium (II), bis(benzonitrile)palladium dichloride, or PdCl₂(dppf) can be used as an alternative. The palladium catalyst can also be on a support material such as an inert organic resin. Typically, the amount of the palladium catalyst used in the reaction mixture is 0.001 to 1 mol % for each mole of monomer, preferably, 0.01 to 1 mol % for each mole of monomer.

The organic solvents suitable for use in the process include those capable of dissolving the monomer to a solution concentration of at least 1 percent, preferably at least 2 percent. Examples of suitable solvents for the process described are hydrocarbons such as hexane, heptane, petroleum ether, cyclohexane, benzene, chlorobenzenes, ethylbenzen, mesitylene, toluene, and xylenes, ethers such as anisole, diethyl ether, tetrahydrofuran, dioxane, dioxolane, diisopropyl ether, dimethoxyethane, t-butyl methyl ether, and diethylene glycol dimethyl ether, ketones such as acetone, methyl ethyl ketone, and isobutyl methyl ketone, alcohols such as methanol, ethanol, propanols, ethylene glycol, and butanols, and amides such as dimethylformamide, dimethylactamide and N-methylpyrrolidone, and the florinated analog thereof, and the mixtures thereof.

The preferred organic solvents include one solvent in which the polymer is soluble. Examples of the preferred solvents are ethers such as tetrahydrofuran, dioxane, dimethyoxyethane, diethylene glycol dimethyl ether, diisopropyl ether, hydrocarbons such as benzene, chlorobenzenes, toluene, xylenes, heptane, and cyclohexane, ketones such as methyl ethyl ketone and isobutyl methyl ketone, amides such as dimethylformamide, dimethylacetamide and N-methylpyrrolidone, and mixtures thereof.

More preferred organic solvents are ethers for example tetrahydrofuran, dimethyoxyethane and dioxane, hydrocarbons for example toluene, chlorobenzenes, and xylenes, and amides for example, dimethylformamide, and dimethylacetamide.

Most preferred organic solvents of the process of the invention are one or more water-insoluble solvents such as toluene or xylenes or tetrahydrofuran, or mixtures thereof. The volume of the solvent of the process of the invention should be maintained at the level for efficient mixing and stirring at reflux as the reaction mixture becomes more viscous with the build-up of polymer molecular weight.

The polymerization reaction mixture may also contain a phase transfer catalyst as disclosed in U.S. Pat. No. 5,777,070. Suitable phase transfer catalysts used in the process of the invention include quaternary ammonium and phosphonium salts, crown ethers and cryptands. Preferably, the phase transfer catalyst is a tetralkylammonium halide, or bisulfate. Examples of the most preferred phase transfer catalyst are tetrabutylammonium chloride and tricaprylylmethylammonium chloride (known as Aliquat® from Aldrich Chemical). The preferred range of the amount of phase transfer catalyst is between 0.01 to 0.5 mole per mole of monomer, more preferably 0.05 to 0.1 mole per mole of monomer.

The polymerization reaction is carried at a temperature of from 0 to 200° C., preferably from 30 to 170° C., and more preferably 50 to 150° C., and most preferably 60 to 120° C. The reaction time is from 1 to 100 hours, preferably 5 to 70 hours, more preferably 5 to 50 hours, and most preferably, 5 to 48 hours.

The process of the present invention can also be extended to the use of monomers in which some or all of the reactive functional groups are not directly attached to the aromatic rings, especially to other kinds of unsaturated monomers.

The synthetic schemes of the compounds according to the present invention are illustrated in Schemes 1-3.

The process of the invention provides conjugated polymers particularly useful for an optical device. The optical device may comprise a luminescent device such as an EL device in which the polymer or small molecules of the present invention is deposited between a cathode and an anode. The polymers or small molecules or the combination thereof can be deposited as thin film by vapor deposition method or from a solution by spin-coating, spray-coating, dip-coating, roller-coating, or ink jet delivery. The thin film may be supported by substrate directly, preferably a transparent substrate, or supported by the substrate indirectly where there is one or more inter layers between the substrate and thin film. The thin film can be used as emitting layer or charge carrier transporting layer.

### General EL Device Architecture:

The present invention can be employed in most organic EL device configurations. These include very simple structures comprising a single anode and cathode to more complex devices, such as passive matrix displays comprised of orthogonal arrays of anodes and cathodes to form pixels, and active-matrix displays where each pixel is controlled independently, for example, with thin film transistors (TFTs).

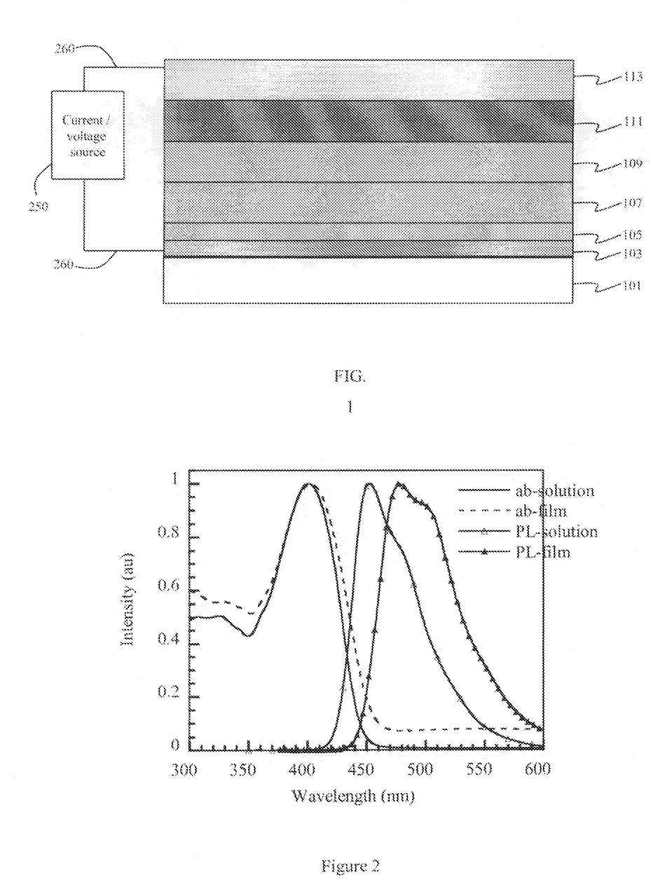

There are numerous configurations of the organic layers wherein the present invention can be successfully practiced. A typical structure is shown in FIG. 1 and includes a substrate 101, an anode 103, a hole-injecting layer 105, a hole-transporting layer 107, a light-emitting layer 109, an electron-transporting layer 111, and a cathode 113. These layers are described in detail below. This figure is for illustration only and the individual layer thickness is not scaled according to the actual thickness. Note that the substrate may alternatively be located adjacent to the cathode, or the substrate may actually constitute the anode or cathode. The organic layers between the anode and cathode are conveniently referred to as the organic EL element. Also, the total combined thickness of the organic layers is preferably less than 500 nm.

The anode and cathode of the OLED are connected to a voltage/current source 250 through electrical conductors 260. The OLED is operated by applying a potential between the anode and cathode such that the anode is at a more positive potential than the cathode. Holes are injected into the organic EL element from the anode and electrons are injected into the organic EL element at the anode. Enhanced device stability can sometimes be achieved when the OLED is operated in an AC mode where, for some time period in the cycle, the potential bias is reversed and no current flows. An example of an AC driven OLED is described in U.S. Pat. No. 5,552,678.

### Substrate:

The OLED device of this invention is typically provided over a supporting substrate 101 where either the cathode or anode can be in contact with the substrate. The electrode in contact with the substrate is conveniently referred to as the bottom electrode. Conventionally, the bottom electrode is the anode, but this invention is not limited to that configuration. The substrate can either be light transmissive or opaque, depending on the intended direction of light emission. The light transmissive property is desirable for viewing the EL emission through the substrate. Transparent glass or plastic is commonly employed in such cases. The substrate may be a complex structure comprising multiple layers of materials. This is typically the case for active matrix substrates wherein TFTs are provided below the EL layers. It is still necessary that the substrate, at least in the emissive pixilated areas, be comprised of largely transparent materials such as glass or polymers. For applications where the EL emission is viewed through the top electrode, the transmissive characteristic of the bottom support is immaterial, and therefore can be light transmissive, light absorbing or light reflective. Substrates for use in this case include, but are not limited to, glass, plastic, semiconductor materials, silicon, ceramics, and circuit board materials. Again, the substrate may be a complex structure comprising multiple layers of materials such as found in active matrix TFT designs. Of course it is necessary to provide in these device configurations a light-transparent top electrode.

### Anode:

When EL emission is viewed through anode 103, the anode should be transparent or substantially transparent to the emission of interest. Common transparent anode materials used in this invention are indium-tin oxide (ITO), indium-zinc oxide (IZO) and tin oxide, but other metal oxides can work including, but not limited to, aluminum- or indium-doped zinc oxide, magnesium-indium oxide, and nickel-tungsten oxide. In addition to these oxides, metal nitrides, such as gallium nitride, and metal selenides, such as zinc selenide, and metal sulfides, such as zinc sulfide, can be used as the anode 103. The anode can be modified with plasma-deposited fluorocarbons. For applications where EL emission is viewed only through the cathode electrode, the transmissive characteristics of anode are immaterial and any conductive material can be used, transparent, opaque or reflective. Example conductors for this application include, but are not limited to, gold, iridium, molybdenum, palladium, and platinum. Typical anode materials, transmissive or otherwise, have a work function of 4.1 eV or greater. Desired anode materials are commonly deposited by any suitable means such as evaporation, sputtering, chemical vapor deposition, or electrochemical means. Anodes can be patterned using well-known photolithographic processes. Optionally, anodes may be polished prior to application of other layers to reduce surface roughness so as to minimize shorts or enhance reflectivity.

### Hole-Injection Layer (HIL):

While not always necessary, it is often useful that a hole-injecting layer 105 be provided between anode 103 and hole-transporting layer 107. The hole-injecting material can serve to improve the film formation property of subsequent organic layers and to facilitate injection of holes into the hole-transporting layer. Suitable materials for use in the hole-injecting layer include, but are not limited to, porphyrinic compounds as described in U.S. Pat. No. 4,720,432, plasma-deposited fluorocarbon polymers as described in U.S. Pat. No. 6,208,075, and some aromatic amines, for example, m-MTDATA (4,4′,4″-tris[(3-methylphenyl)phenylamino]triphenylamine). Alternative hole-injecting materials reportedly useful in organic EL devices are described in EP 0 891 121 A1 and EP 1 029 909 A1.

### Hole-Transporting Layer (HTL)

The hole-transporting layer 107 of the organic EL device in general contains at least one hole-transporting compound such as an aromatic tertiary amine, where the latter is understood to be a compound containing at least one trivalent nitrogen atom that is bonded only to carbon atoms, at least one of which is a member of an aromatic ring. In one form the aromatic tertiary amine can be an arylamine, such as a monoarylamine, diarylamine, triarylamine, or a polymeric arylamine. Exemplary monomeric triarylamines are illustrated by Klupfel et al. U.S. Pat. No. 3,180,730. Other suitable triarylamines substituted with one or more vinyl radicals and/or including at least one active hydrogen containing group are disclosed by Brantley et al U.S. Pat. Nos. 3,567,450 and 3,658,520.

A more preferred class of aromatic tertiary amines are those which include at least two aromatic tertiary amine moieties as described in U.S. Pat. Nos. 4,720,432 and 5,061,569. Such compounds include those represented by structural formula (A).

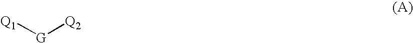

wherein Q₁ and Q₂ are independently selected aromatic tertiary amine moieties and G is a linking group such as an arylene, cycloalkylene, or alkylene group of a carbon to carbon bond. In one embodiment, at least one of Q₁ or Q₂ contains a polycyclic fused ring structure, e.g., a naphthalene. When G is an aryl group, it is conveniently a phenylene, biphenylene, or naphthalene moiety.

A useful class of triarylamines satisfying structural formula (A) and containing two triarylamine moieties is represented by structural formula (B):

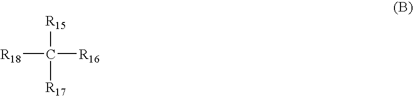

wherein:

R₁₅ and R₁₆ each independently represents a hydrogen atom, an aryl group, or an alkyl group or R₁ and R₂ together represent the atoms completing a cycloalkyl group; and

R₁₇ and R₁₈ each independently represents an aryl group, which is in turn substituted with a diaryl substituted amino group, as indicated by structural formula (C):

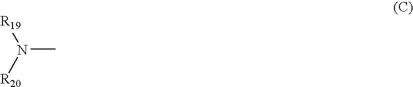

wherein R₁₉ and R₂₀ are independently selected aryl groups. In one embodiment, at least one of R₁₉ or R₂₀ contains a polycyclic fused ring structure, e.g., a naphthalene.

Another class of aromatic tertiary amines are the tetraaryldiamines. Desirable tetraaryldiamines include two diarylamino groups, such as indicated by formula (C), linked through an arylene group. Useful tetraaryldiamines include those represented by formula (D):

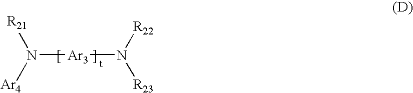

wherein
each Ar₃ is an independently selected arylene group, such as a phenylene or anthracene moiety,
t is an integer of from 1 to 4, and
Ar₄, R₂₁, R₂₂, and R₂₃ are independently selected aryl groups.

In a typical embodiment, at least one of Ar₄, R₂₁, R₂₂, and R₂₃ is a polycyclic fused ring structure, e.g., a naphthalene.

The various alkyl, alkylene, aryl, and arylene moieties of the foregoing structural formulae (A), (B), (C), (D), can each in turn be substituted. Typical substituents include alkyl groups, alkoxy groups, aryl groups, aryloxy groups, and halogen such as fluoride, chloride, and bromide. The various alkyl and alkylene moieties typically contain from about 1 to 6 carbon atoms. The cycloalkyl moieties can contain from 3 to about 10 carbon atoms, but typically contain five, six, or seven ring carbon atoms—e.g., cyclopentyl, cyclohexyl, and cycloheptyl ring structures. The aryl and arylene moieties are usually phenyl and phenylene moieties.

The hole-transporting layer can be formed of a single or a mixture of aromatic tertiary amine compounds. Specifically, one may employ a triarylamine, such as a triarylamine satisfying the formula (B), in combination with a tetraaryldiamine, such as indicated by formula (D). When a triarylamine is employed in combination with a tetraaryldiamine, the latter is positioned as a layer interposed between the triarylamine and the electron injecting and transporting layer. Illustrative of useful aromatic tertiary amines are the following:
1,1-Bis(4-di-p-tolylaminophenyl)cyclohexane
1,1-Bis(4-di-p-tolylaminophenyl)-4-phenylcyclohexane
4,4′-Bis(diphenylamino)quadriphenyl
Bis(4-dimethylamino-2-methylphenyl)-phenylmethane
N,N,N-Tri(p-tolyl)amine
4-(di-p-tolylamino)-4′-[4(di-p-tolylamino)-styryl]stilbene
N,N,N′,N′-Tetra-p-tolyl-4-4″-diaminobiphenyl
N,N,N′,N′-Tetraphenyl-4,4′-diaminobiphenyl
N,N,N′,N′-tetra-1-naphthyl-4,4′-diaminobiphenyl
N,N,N′,N′-tetra-2-naphthyl-4,4′-diaminobiphenyl
N-Phenylcarbazole
4,4′-Bis[N-(1-naphthyl)-N-phenylamino]biphenyl
4,4′-Bis[N-(1-naphthyl)-N-(2-naphthyl)amino]biphenyl
4,4″-Bis[N-(1-naphthyl)-N-phenylamino]ₚ-terphenyl
4,4′-Bis[N-(2-naphthyl)-N-phenylamino]biphenyl
4,4′-Bis[N-(3-acenaphthenyl)-N-phenylamino]biphenyl
1,5-Bis[N-(1-naphthyl)-N-phenylamino]naphthalene
4,4′-Bis[N-(9-anthryl)-N-phenylamino]biphenyl
4,4″-Bis[N-(1-anthryl)-N-phenylamino]-p-terphenyl
4,4′-Bis[N-(2-phenanthryl)-N-phenylamino]biphenyl
4,4′-Bis[N-(8-fluoranthenyl)-N-phenylamino]biphenyl
4,4′-Bis[N-(2-pyrenyl)-N-phenylamino]biphenyl
4,4′-Bis[N-(2-naphthacenyl)-N-phenylamino]biphenyl
4,4′-Bis[N-(2-perylenyl)-N-phenylamino]biphenyl
4,4′-Bis[N-(1-coronenyl)-N-phenylamino]biphenyl
2,6-Bis(di-p-tolylamino)naphthalene
2,6-Bis[di-(1-naphthyl)amino]naphthalene
2,6-Bis[N-(1-naphthyl)-N-(2-naphthyl)amino]naphthalene
N,N,N′,N′-Tetra(2-naphthyl)-4,4″-diamino-p-terphenyl
4,4′-Bis {N-phenyl-N-[4-(1-naphthyl)-phenyl]amino} biphenyl
4,4′-Bis[N-phenyl-N-(2-pyrenyl)amino]biphenyl
2,6-Bis[N,N-di(2-naphthyl)amine]fluorene
1,5-Bis[N-(1-naphthyl)-N-phenylamino]naphthalene
4,4′,4″-tris [(3-methylphenyl)phenylamino]triphenylamine

Another class of useful hole-transporting materials includes polycyclic aromatic compounds as described in EP 1 009 041. Tertiary aromatic amines with more than two amine groups may be used including oligomeric materials. In addition, polymeric hole-transporting materials can be used such as poly(N-vinylcarbazole) (PVK), polythiophenes, polypyrrole, polyaniline (Yang, Y. et al. Appl. Phys. Lett. 1994, 64, 1245) and copolymers such as poly(3,4-ethylenedioxythiophene)/poly(4-styrenesulfonate) also called PEDOT/PSS(Groenendaal, L. B. et al. Adv. Mater. 2000, 12, 481).

### Light-Emitting Layer (LEL)

As more fully described in commonly-assigned U.S. Pat. Nos. 4,769,292 and 5,935,721, the light-emitting layer (LEL) 109 of the organic EL element includes a luminescent or fluorescent material where electroluminescence is produced as a result of electron-hole pair recombination in this region. The light-emitting layer can be comprised of a single material including both small molecules and polymers, but more commonly consists of a host material doped with a guest compound or compounds where light emission comes primarily from the dopant and can be of any color. The host materials in the light-emitting layer can be an electron-transporting material, as defined below, a hole-transporting material, as defined above, or another material or combination of materials that support hole-electron recombination. The dopant is usually chosen from highly fluorescent dyes, but phosphorescent compounds, e.g., transition metal complexes as described in WO 98/55561, WO 00/18851, WO 00/57676, and WO 00/70655 are also useful. Simultaneously, the color of the EL devices can be tuned using dopants of different emission wavelengths. By using a mixture of dopants, EL color characteristics of the combined spectra of the individual dopant are produced. This dopant scheme has been described in considerable detail for EL devices in commonly-assigned U.S. Pat. No. 4,769,292 for fluorescent dyes. Dopants are typically coated as 0.01 to 10% by weight into the host material. Polymeric materials such as polyfluorenes and poly(arylene vinylenes) (e.g., poly(p-phenylenevinylene), PPV) can also be used as the host material. In this case, small molecule dopants can be molecularly dispersed into the polymeric host, or the dopant could be added by copolymerizing a minor constituent into the host polymer.

An important relationship for choosing a dye as a dopant is a comparison of the bandgap potential which is defined as the energy difference between the highest occupied molecular orbital (HOMO) and the lowest unoccupied molecular orbital (LUMO) of the molecule. For efficient energy transfer from the host to the dopant molecule, a necessary condition is that the band gap of the dopant is smaller than that of the host material. For phosphorescent emitters it is also important that the host triplet energy level of the host be high enough to enable energy transfer from host to dopant.

For small molecules, host and emitting molecules known to be of use include, but are not limited to, those disclosed in U.S. Pat. Nos. 4,768,292; 5,141,671; 5,150,006; 5,151,629; 5,405,709; 5,484,922; 5,593,788; 5,645,948; 5,683,823; 5,755,999; 5,928,802; 5,935,720; 5,935,721, and 6,020,078.

For example, small molecule metal complexes of 8-hydroxyquinoline and similar derivatives (Formula E) constitute one class of useful host compounds capable of supporting electroluminescence, and are particularly suitable for light emission of wavelengths longer than 500 nm, e.g., green, yellow, orange, and red.

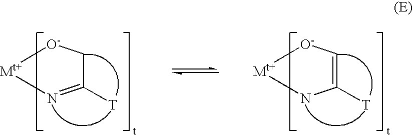

wherein:

M represents a metal;

t is an integer of from 1 to 4; and

T independently in each occurrence represents the atoms completing a nucleus having at least two fused aromatic rings.

From the foregoing it is apparent that the metal can be monovalent, divalent, trivalent, or tetravalent metal. The metal can, for example, be an alkali metal, such as lithium, sodium, or potassium; an alkaline earth metal, such as magnesium or calcium; an earth metal, such aluminum or gallium, or a transition metal such as zinc or zirconium. Generally any monovalent, divalent, trivalent, or tetravalent metal known to be a useful chelating metal can be employed.

T completes a heterocyclic nucleus containing at least two fused aromatic rings, at least one of which is an azole or azine ring. Additional rings, including both aliphatic and aromatic rings, can be fused with the two required rings, if required. To avoid adding molecular bulk without improving on function the number of ring atoms is usually maintained at 18 or less.

Illustrative of useful chelated oxinoid compounds are the following:
CO-1: Aluminum trisoxine[alias, tris(8-quinolinolato)aluminum(III)]
CO-2: Magnesium bisoxine[alias, bis(8-quinolinolato)magnesium(I1)]
CO-3: Bis[benzo{f}-8-quinolinolato]zinc (II)
CO-4: Bis(2-methyl-8-quinolinolato)aluminum(III)-μ-oxo-bis(2-methyl-8-quinolinolato) aluminum(III)
CO-5: Indium trisoxine[alias, tris(8-quinolinolato)indium]
CO-6: Aluminum tris(5-methyloxine) [alias, tris(5-methyl-8-quinolinolato) aluminum(III)]
CO-7: Lithium oxine[alias, (8-quinolinolato)lithium(I)]
CO-8: Gallium oxine[alias, tris(8-quinolinolato)gallium(III)]
CO-9: Zirconium oxine[alias, tetra(8-quinolinolato)zirconium(IV)]

Derivatives of 9,10-di-(2-naphthyl)anthracene (Formula F) constitute one class of useful hosts capable of supporting electroluminescence, and are particularly suitable for light emission of wavelengths longer than 400 nm, e.g., blue, green, yellow, orange or red.

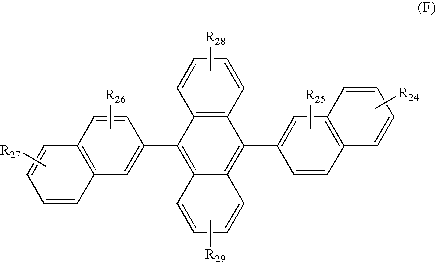

wherein: R₂₄, R₂₅, R₂₆, R₂₇, R₂₈, and R₂₉ represent one or more substituents on each ring where each substituent is individually selected from the following groups:

Group 1: hydrogen, or alkyl of from 1 to 24 carbon atoms;

Group 2: aryl or substituted aryl of from 5 to 20 carbon atoms;

Group 3: carbon atoms from 4 to 24 necessary to complete a fused aromatic ring of anthracenyl; pyrenyl, or perylenyl;

Group 4: heteroaryl or substituted heteroaryl of from 5 to 24 carbon atoms as necessary to complete a fused heteroaromatic ring of furyl, thienyl, pyridyl, quinolinyl or other heterocyclic systems;

Group 5: alkoxylamino, alkylamino, or arylamino of from 1 to 24 carbon atoms; and

Group 6: fluorine, chlorine, bromine or cyano.

Illustrative examples include 9,10-di-(2-naphthyl)anthracene and 2-t-butyl-9,10-di-(2-naphthyl)anthracene. Other anthracene derivatives can be useful as a host in the LEL, including derivatives of 9,10-bis[4-(2,2-diphenylethenyl)phenyl]anthracene.

Benzazole derivatives (Formula G) constitute another class of useful hosts capable of supporting electroluminescence, and are particularly suitable for light emission of wavelengths longer than 400 nm, e.g., blue, green, yellow, orange or red.

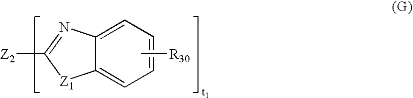

wherein:

t₁ is an integer of 3 to 8;

Z₁ is O, NR₃₁ or S; and

R₃₀ and R₃₁ are individually hydrogen; alkyl of from 1 to 24 carbon atoms, for example, propyl, t-butyl, heptyl, and the like; aryl or hetero-atom substituted aryl of from 5 to 20 carbon atoms for example phenyl and naphthyl, furyl, thienyl, pyridyl, quinolinyl and other heterocyclic systems; or halo such as chloro, fluoro; or atoms necessary to complete a fused aromatic ring;

Z₂ is a linkage unit consisting of alkyl, aryl, substituted alkyl, or substituted aryl, which conjugately or unconjugately connects the multiple benzazoles together. An example of a useful benzazole is 2,2′,2″-(1,3,5-phenylene)tris[1-phenyl-1H-benzimidazole].

Distyrylarylene derivatives are also useful hosts, as described in U.S. Pat. No. 5,121,029. Carbazole derivatives are particularly useful hosts for phosphorescent emitters.

Polymers incorporating the above small molecule moieties as represented by formulas (E), (F), and (G) are useful host materials. Examples of 9,10-di-(2-naphthyl)anthracene-containing polymers are disclosed in U.S. Pat. No. 6,361,887.

Useful fluorescent dopants (FD) include, but are not limited to, derivatives of anthracene, tetracene, xanthene, perylene, rubrene, coumarin, rhodamine, and quinacridone, dicyanomethylenepyran compounds, thiopyran compounds, polymethine compounds, pyrilium and thiapyrilium compounds, fluorene derivatives, periflanthene derivatives, indenoperylene derivatives, bis(azinyl)amine boron compounds, bis(azinyl)methane compounds, and carbostyryl compounds. Useful phosphorescent dopants (PD) include but are not limited to organometallic complexes of transition metals of iridium, platinum, palladium, or osmium. Illustrative examples of useful dopants include, but are not limited to, the following:

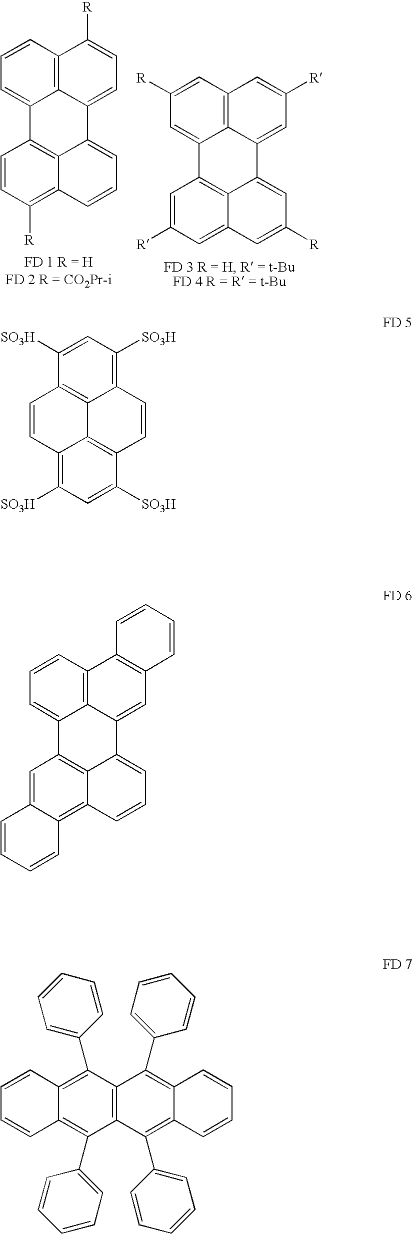

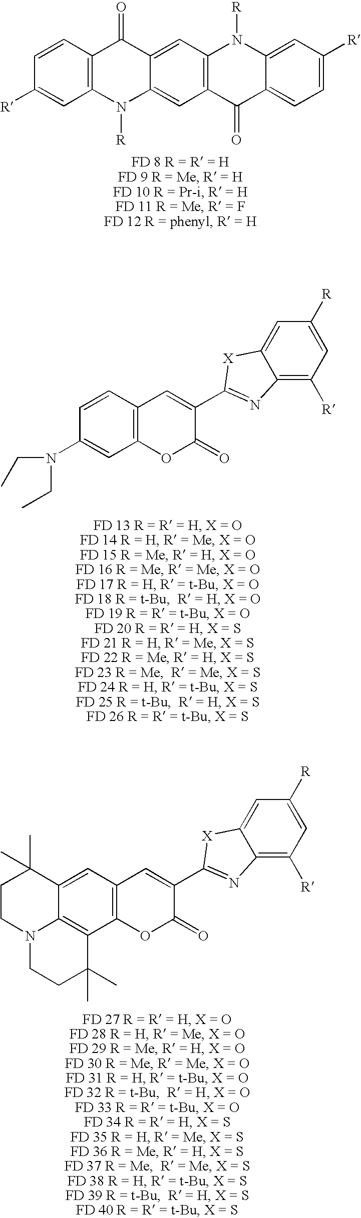

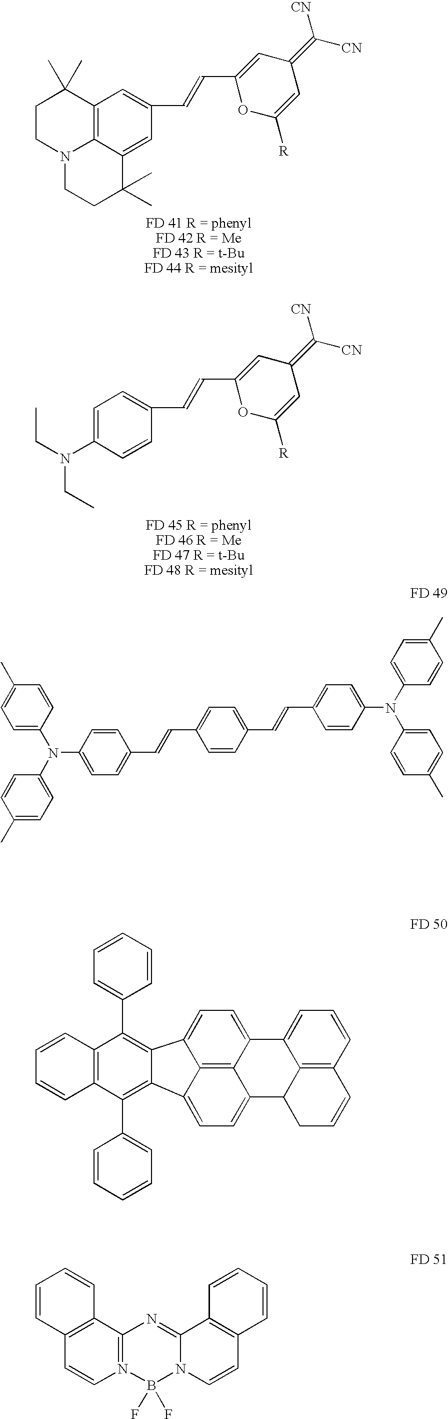

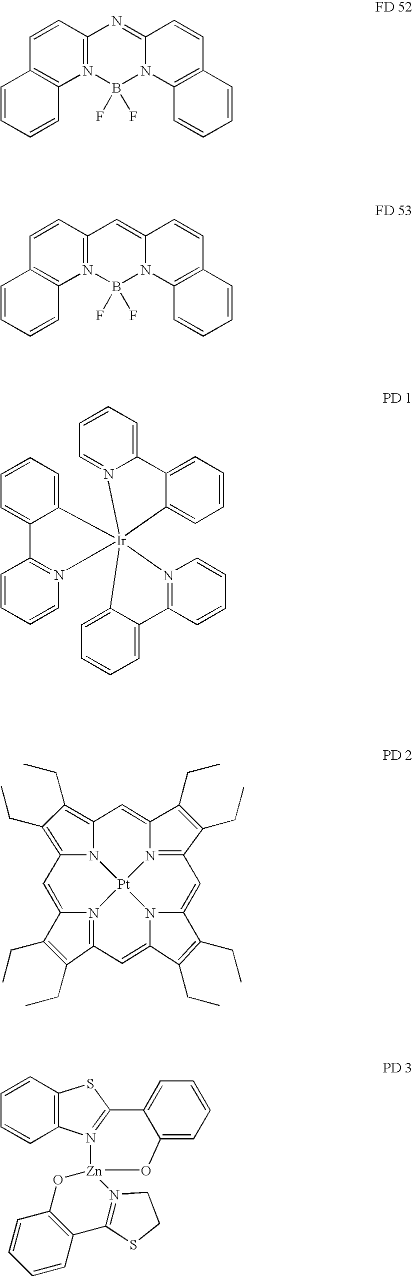

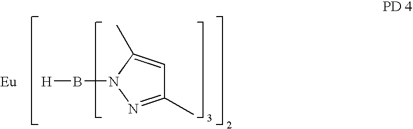

### Electron-Transporting Layer (ETL):

Preferred thin film-forming materials for use in forming the electron-transporting layer 111 of the organic EL devices of this invention are metal chelated oxinoid compounds, including chelates of oxine itself (also commonly referred to as 8-quinolinol or 8-hydroxyquinoline). Such compounds help to inject and transport electrons and exhibit both high levels of performance and are readily fabricated in the form of thin films. Exemplary of contemplated oxinoid compounds are those satisfying structural formula (E), previously described.

Other electron-transporting materials include various butadiene derivatives as disclosed in U.S. Pat. No. 4,356,429 and various heterocyclic optical brighteners as described in U.S. Pat. No. 4,539,507. Benzazoles satisfying structural formula (G) are also useful electron transporting materials. Triazines are also known to be useful as electron transporting materials. Oxadiazole compounds including small molecules and polymers are useful electron transporting materials as described in U.S. Pat. No. 6,451,457.

### Cathode:

When light emission is viewed solely through the anode, the cathode 113 used in this invention can be comprised of nearly any conductive material. Desirable materials have good film-forming properties to ensure good contact with the underlying organic layer, promote electron injection at low voltage, and have good stability. Useful cathode materials often contain a low work function metal (<4.0 eV) or metal alloy. One preferred cathode material is comprised of a Mg:Ag alloy wherein the percentage of silver is in the range of 1 to 20%, as described in commonly-assigned U.S. Pat. No. 4,885,211. Another suitable class of cathode materials includes bilayers comprising a thin electron-injection layer (EIL) in contact with the organic layer (e.g., ETL) which is capped with a thicker layer of a conductive metal. Here, the EIL preferably includes a low work function metal or metal salt, and if so, the thicker capping layer does not need to have a low work function. One such cathode is comprised of a thin layer of LiF followed by a thicker layer of Al as described in commonly-assigned U.S. Pat. No. 5,677,572. Other useful cathode material sets include, but are not limited to, those disclosed in commonly-assigned U.S. Pat. Nos. 5,059,861; 5,059,862, and 6,140,763.

When light emission is viewed through the cathode, the cathode must be transparent or nearly transparent. For such applications, metals must be thin or one must use transparent conductive oxides, or a combination of these materials. Optically transparent cathodes have been described in more detail in U.S. Pat. Nos. 4,885,211; 5,247,190; 5,703,436; 5,608,287; 5,837,391; 5,677,572; 5,776,622; 5,776,623; 5,714,838; 5,969,474; 5,739,545; 5,981,306; 6,137,223; 6,140,763; 6,172,459; 6,278,236; 6,284,3936; EP 1 076 368 and JP 3,234,963. Cathode materials are typically deposited by evaporation, sputtering, or chemical vapor deposition. When needed, patterning can be achieved through many well known methods including, but not limited to, through-mask deposition, integral shadow masking as described in U.S. Pat. No. 5,276,380 and EP 0 732 868, laser ablation, and selective chemical vapor deposition. Other Useful Organic Layers and Device Architecture

In some instances, layers 109 and 111 can optionally be collapsed into a single layer that serves the function of supporting both light emission and electron transportation. Alternatively, layers 107, 109 and 111 can optionally be collapsed into a single layer that serves the function of supporting both light emission and hole and electron transportation. This is the preferred EL device structure of this invention and is referred to as “single-layer” device.

It also known in the art that emitting dopants may be added to the hole-transporting layer, which may serve as a host. Multiple dopants may be added to one or more layers in order to create a white-emitting EL device, for example, by combining blue- and yellow-emitting materials, cyan- and red-emitting materials, or red-, green-, and blue-emitting materials. White-emitting devices are described, for example, in EP 1 187 235, EP 1 182 244, U.S. Patent Publication 20020025419, and U.S. Pat. Nos. 5,683,823; 5,503,910; 5,405,709; and 5,283,182.

Additional layers such as electron or hole-blocking layers as taught in the art may be employed in devices of this invention. Hole-blocking layers are commonly used to improve efficiency of phosphorescent emitter devices, for example, as in U.S. Patent Publication 20020015859.

This invention may be used in so-called stacked device architecture, for example, as taught in U.S. Pat. Nos. 5,703,436 and 6,337,492.

### Deposition of Organic Layers

The organic materials mentioned above can be deposited as high quality transparent thin films by various methods such as a vapor deposition or sublimation method, an electron-beam method, a sputtering method, a thermal transferring method, a molecular lamination method and a coating method such as solution casting, spin-coating or inkjet printing, with an optional binder to improve film formation. If the material is a polymer, solvent deposition is usually preferred. The material to be deposited by sublimation can be vaporized from a sublimator “boat” often include a tantalum material, e.g., as described in U.S. Pat. No. 6,237,529, or can be first coated onto a donor sheet and then sublimed in closer proximity to the substrate. Layers with a mixture of materials can utilize separate sublimator boats or the materials can be pre-mixed and coated from a single boat or donor sheet. Patterned deposition can be achieved using shadow masks, integral shadow masks (U.S. Pat. No. 5,294,870), spatially-defined thermal dye transfer from a donor sheet (U.S. Pat. Nos. 5,688,551; 5,851,709 and 6,066,357) and inkjet method (U.S. Pat. No. 6,066,357).

Preferably, the spin-coating or inkjet printing technique is used to deposit the organic material of the invention, only one compound is deposited in a single layer device.

### Encapsulation:

Most organic EL devices are sensitive to moisture or oxygen, or both, so they are commonly sealed in an inert atmosphere such as nitrogen or argon, along with a desiccant such as alumina, bauxite, calcium sulfate, clays, silica gel, zeolites, alkaline metal oxides, alkaline earth metal oxides, sulfates, or metal halides and perchlorates. Methods for encapsulation and desiccation include, but are not limited to, those described in U.S. Pat. No. 6,226,890. In addition, barrier layers such as SiOx, Teflon, and alternating inorganic/polymeric layers are known in the art for encapsulation.

### Optical Optimization:

Organic EL devices of this invention can employ various well-known optical effects in order to enhance its properties if desired. This includes optimizing layer thicknesses to yield maximum light transmission, providing dielectric mirror structures, replacing reflective electrodes with light-absorbing electrodes, providing anti glare or anti-reflection coatings over the display, providing a polarizing medium over the display, or providing colored, neutral density, or color conversion filters over the display. Filters, polarizers, and anti-glare or anti-reflection coatings may be specifically provided over the cover or as part of the cover.

### EXAMPLES

The invention and its advantages are further illustrated by the following specific examples:

### Synthesis of Small Molecules

The monomers to be used in the present invention to construct polymers are not necessary to be particularly restricted. Any monomers can be used as long as the polymer formed is a polymer which satisfies the general formulas (V) and (VI). Typical synthesis is illustrated in Schemes 1.

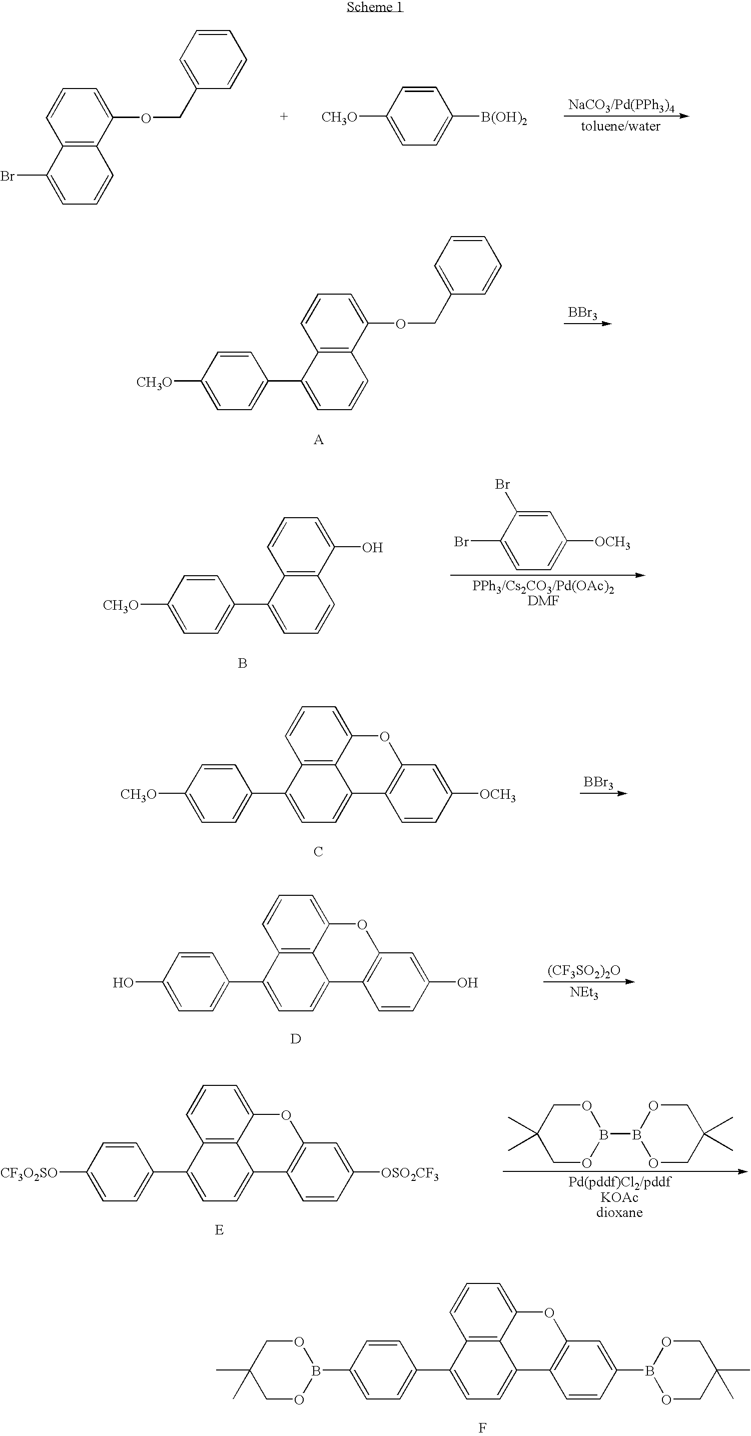

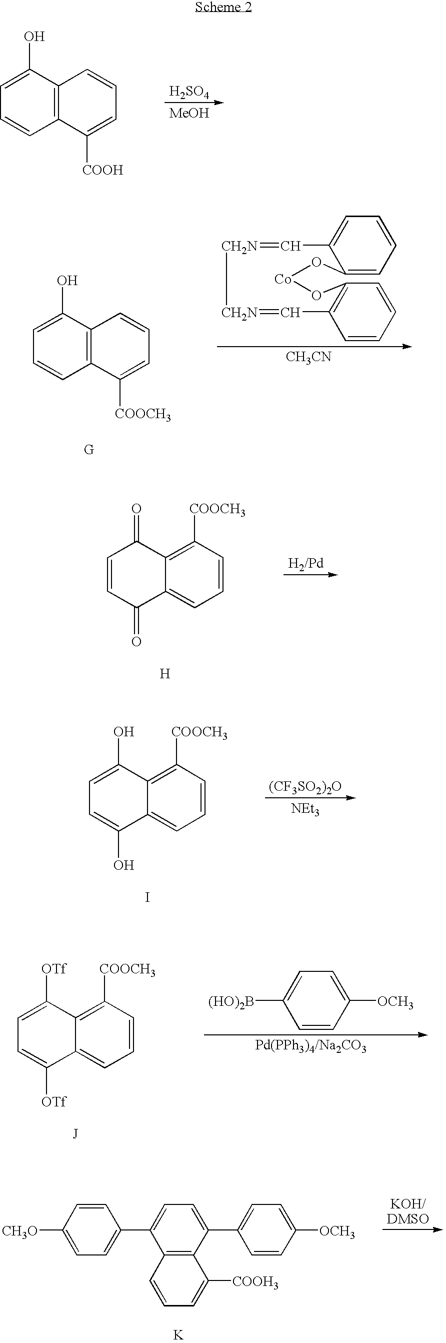

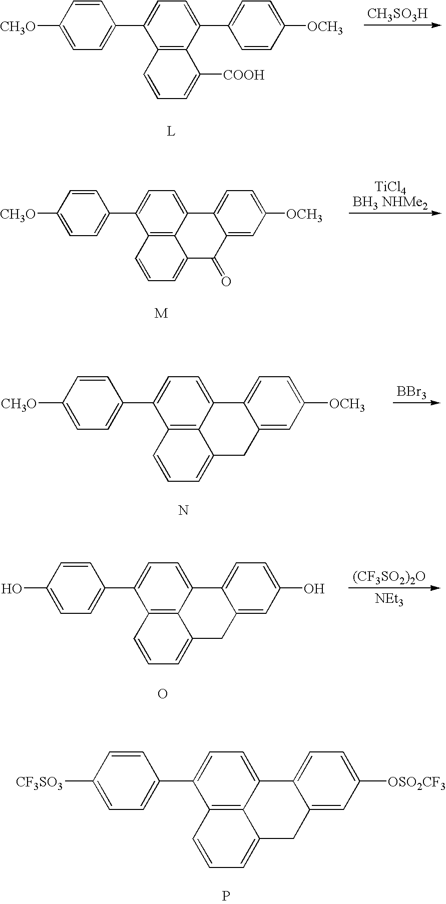

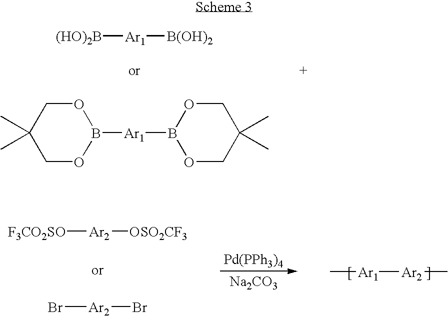

### Example 1

### Synthesis of Compound A

1-Bromo-5-benzyloxynaphthalene (10.0 g, 0.034 mol), 4-methoxyphenyl boronic acid (5.6 g, 0.037 mol), sodium carbonate aqueous solution (2 M, 13.6 mL), 2 drops of phase transfer reagent Aliquat 336, and toluene (100 mL) were mixed in a 250-mL round bottomed flask and degassed with nitrogen for 20 min. Catalyst Pd(PPh₃)₄ (0.02 mol %) was added and the reaction was heated to 90° C. overnight. The reaction was cooled down and extracted with ethyl acetate. The organic phase was dried over magnesium sulfate and concentrated. The crude product was purified by column on passing through a short silica gel column using methylene chloride as an eluent and recrystallized from heptane/ethanol to give 9.5 g of pure product as yellow solid (83% yield). ¹H NMR (CDCl₃) δ ppm: 3.92 (s, 3H), 5.31 (s, 2H), 6.93-6.96 (m, 1H), 7.06-7.09 (m, 2H), 7.36-7.58 (m, 11H), 8.44-8.49 (m, 1H); ¹³C NMR (CDCl₃) δ ppm: 55.28, 70.15, 105.03, 113.60, 118.66, 121.40, 124.76, 125.68, 126.13, 127.33, 127.56, 127.89, 128.56, 131.10, 132.93, 133.42, 137.14, 139.50, 154.60, 158.83. FD-MS: 340 (M⁺).

### Example 2

### Synthesis of Compound B

Compound A (9.0 g, 0.030 mol) was dissolved in methylene chloride and cooled to 0° C. for 20 min. To the solution was added boron tribromide (1 M in methylene chloride, 46 mL) dropwise. The reaction was stirred for 20 min. and quenched with saturated Na₂CO₃ solution, and extracted with methylene chloride. The crude product was purified by column chromatography on silica gel using 1/1 methylene chloride/heptane as an eluent to 3.8 g give pure product as light brown solid (50% yield). ¹H NMR (CDCl₃) δ ppm: 3.89 (s, 3H), 6.81 (d, J=7.2 Hz, 1H), 7.02 (d, J=8.6 Hz, 2H), 7.24 (t, J=7.3 Hz, 1H), 7.41 (d, J=8.5 Hz, 3H), 7.51 (t, J=7.5 Hz, 2H), 8.20 (d, J=8.3 Hz, 1H); ¹³C NMR (CDCl₃) δ ppm: 55.35, 108.41, 112.96, 113.64, 118.91, 120.82, 124.74, 125.66, 127.54, 127.82, 128.76, 130.33, 131.08, 151.54, 158.82; FD-MS: 250 (M⁺).

### Example 3

### Synthesis of Compound C

Compound B (2.8 g, 0.011 mol), 3,4-dibromoanisole (2.9 g, 0.0111 mol), cesium carbonate (5.6 g, 0.017 mol), triphenylphosphine (0.59 g, 0.002 mol) were dissolved in DMF and degassed with nitrogen for 20 min. Catalyst palladium acetate (0.125 g, 0.006 mol) was added and the reaction was heated to 160° C. overnight. Reaction was cooled down and water was added. The reaction was extracted with ether and the crude product was purified by column chromatography on silica gel using 1/9 ether/heptane as an eluent to give 1.0 g pure product as light green fluorescent solid (26% yield). ¹H NMR (CDCl₃) δ ppm: 3.84 (s, 3H), 3.88 (s, 3H), 6.63-6.71 (m, 2H), 6.90-7.02 (m, 3H), 7.27-7.52 (m, 6H), 7.73 (d, J=8.7 Hz, 1H); ¹³C NMR (CDCl₃): δ5.32, 55.46, 101.36, 107.94, 110.69, 112.91, 113.83, 117.86, 118.28, 123.67, 127.02, 128.32, 130.64, FD-MS: 354 (M⁺).

### Example 4

### Synthesis of Compound D

Compound C (5.0 g, 0.014 mol) was dissolved in methylene chloride and cooled to 0° C. for 20 min. To the solution was added boron tribromide (1 M in methylene chloride, 42 mL) dropwise. The reaction was stirred for 4 h and quenched with saturated Na₂CO₃ solution, and extracted with ethyl acetate. The crude product was purified by column chromatography on silica gel using 4/6 ether/heptane as an eluent to 4.0 g give pure product as light brown solid (88% yield). FD-MS: 326 (M⁺).

### Example 5

### Synthesis of Compound E

Compound D (4.5 g, 0.014 mol) was dissolved in 100 mL of methylene chloride and triethylamine (3.1 g, 0.030 mol) was added. The reaction was cooled to 0° C. and triflic anhydride (8.6 g, 0.030 mol) was added slowly. The reaction was stirred at room temperature for a few hours and quenched with water. The reaction was quenched with water and extracted with ether and the crude product was purified by column chromatography on silica gel using 5/95 methylene chloride/heptane as an eluent to give 3.5 g pure product as light green fluorescent solid (43% yield). FD-MS: 590 (M⁺).

### Example 6

### Synthesis of Compound F

Compound E (3.3 g, 0.0064 mol), bis(neopentyl glycola)diboron (3.2 g, 0.014 mol) and potassium acetate (3.8 g, 0.039 mol) were mixed in 70 mL of dioxane. The mixture was bubbled with nitrogen for 15 min and catalyst bis(diphenylphosphino)ferrocene palladium chloride (Pd(dppf)₂Cl₂) (160 mg, 0.03 mol %) and ligand dppf (110 mg, 0.03 mol %) were added. The reaction was heated to 80° C. under nitrogen overnight. The reaction was extracted with methylene chloride and water, and the crude product was passed through a short column of silica gel using 1/1 methylene chloride/heptane as an eluent. The product was recrystallized from methylene chloride/heptane to give 1.8 g of pure product as bright yellow fluorescent solid (46% yield). ¹H NMR (CDCl₃): 1.03(s, 6H), 1.05 (s, 6H), 3.78 (s, 4H), 3.80 (s, 4H), 6.92-7.93 (m, 12H); ¹³C NMR (CDCl₃): 21.91, 31.90, 72.34, 108.17, 114.24, 114.317, 116.24, 117.97, 121.78, 122.35, 126.57, 127.11, 127.26, 128.20, 128.28, 128.62, 128.79, 941.12, 133.10, 133.95, 138.40, 142.87, 142.89; FD-MS: 518 (M⁺).

### Example 7

### Synthesis of Compound G

5-Hydroxy-1-naphtholic acid (5.0 g, 0.027 mol) was dissolved in 50 mL of methanol and 0.1 mL of concentrated sulfuric acid was added. The reaction was heated overnight under reflux. Methanol was removed and the reaction was extracted with water and methylene chloride. The crude product was purified by column chromatography using 3/7 ether/heptante to give 5.1 g of pure product (95% yield). FD-MS: 202 (M⁺).

### Example 8

### Synthesis of Compound H

Compound G (1.6 g, 0.006 mol) was dissolved in 15 mL of acetonitrile and 0.25 g of cobalt catalyst was added. The reaction was bubbled under air overnight at room temperature. Solvent was removed and the crude product was purified by column chromatography using 3/7 ether/heptante to give 0.46 g of pure product as off white solid (27% yield). ¹H NMR (CDCl₃): 3.96 (s, 3H), 6.96 (s, 2H), 7.63-7.66 (m, 1H), 7.74-7.88 (m, 1H), 8.11-8.14 (m, 1H); FD-MS: 216 (M⁺).

### Example 9

### Synthesis of Compound I

Compound G (4.3 g, 0.02 mol) was dissolved in 50 mL of THF and 0.21 g of Pd/C was added. The reaction was hydrogenated for 2 hours, passed through a pad of celite and concentrated. The crude product as dark brown oil was used without further purification for next step. ¹H NMR (CDCl₃): 4.05 (s, 3 H), 5.82 (s, 1H), 6.82 (d, J=8.2 Hz, 1H0, 6.99 (d, J=8.2 Hz, 1H), 7.44 (t, J=7.6 Hz, 1H), 8.20 (d, J=7.3 Hz, 1H), 8.53 (d, J=7.3 Hz, 1H), 9.36 (s, 1H); FD-MS: 218 (M⁺).

### Example 10

### Synthesis of Compound J

Compound I (12.8 g, 0.059 mol) was dissolved in 200 mL of methylene chloride and triethylamine (14.9 g, 0.15 mol) was added. The reaction was cooled to 0° C. and triflic anhydride (41.6 g, 0.15 mol) was added slowly. The reaction was stirred at room temperature overnight and quenched with water. The reaction was extracted with ether and the crude product was purified by column chromatography on silica gel using to give 9.0 g pure product as light brown solid (32% yield). ¹H NMR (CDCl₃): 4.02 (s, 3H), 5.82 (s, 1H), 7.58 (d, J=8.4 Hz, 1H0, 7.66 (d, J=8.5 Hz, 1H), 7.77 (t, J=7.2 Hz, 1H), 7.87 (d, J=7.1 Hz, 1H), 8.23 (d, J=8.5 Hz, 1H) FD-MS: 482 (M⁺).

### Example 11

### Synthesis of Compound K

Compound J (7.7 g, 0.016 mol), 4-methoxyphenyl boronic acid (5.3 g, 0.035 mol), sodium carbonate aqueous solution (2 M, 26 mL), 2 drops of phase transfer reagent Aliquat 336, and toluene (100 mL) were mixed in a 250-mL round bottomed flask and degassed with nitrogen for 20 min. Catalyst Pd(PPh₃)₄ (0.03 mol %) was added and the reaction was heated to 90° C. overnight. The reaction was cooled down and extracted with ethyl acetate. The organic phase was dried over magnesium sulfate and concentrated. The crude product was purified by column on passing through a short silica gel column to give 3.1 g of pure product as white crystalline solid (49% yield). ¹H NMR (CDCl₃) δ ppm: 3.20 (s, 3H), 3.83 (s, 3H), 3.86 (s, 3H), 6.95-7.03 (m, 4H), 7.33-7.45 (m, 7H), 7.68 (d, J=7.2 Hz, 1H), 8.05 (d, J=8.5 Hz, 1H); ¹³C NMR (CDCl₃) δ ppm: 52.14, 55.66, 114.10, 114.14, 124.82, 127.51, 128.94, 129.18, 130.11, 130.16, 130.36, 131.59, 131.97, 133.13, 133.58, 135.75, 138.77, 139.75, 159.07, 159.42, 170.70; FD-MS: 398 (M⁺).

### Example 12

### Synthesis of Compound L

Compound K (2.9 g, 0.0073 mol) was dissolved in 30 mL of DMSO and ground KOH (3.0 g, 0.054 mol) was added and the reaction was heated to 90° C. overnight. The reaction was cooled down and poured into 2 N HCl solution. Off-white precipitate formed and filtered to give 2.3 g (quantitative yield) of off-white solid. ¹H NMR (CDCl₃) δ ppm: 3.79 (s, 3H), 3.84 (s, 3H), 6.94 (d, J=8.5 Hz, 2H), 7.10 (d, J=8.5 Hz, 2H), 7.26 (d, J=8.5 Hz, 2H), 7.39-7.52 (m, 5H), 7.66 (d, J=7.2 Hz, 1H), 7.94 (d, J=8.5 Hz, 1H); ¹³C NMR (CDCl₃) δ ppm: 55.03, 55.22, 113.60, 114.03, 125.02, 126.98, 127.89, 128.22, 128.41, 129.33, 129.86, 131.10, 132.10, 132.53, 133.36, 135.03, 138.61, 158.37, 158.80, 170.01; FD-MS: 384 (M⁺).

### Example 13

### Synthesis of Compound M

Compound L (3.8 g, 0.0099 mol) was dissolved in 30 mL of methane sulfonic acid and heated to 70° C. for 15 min. The reaction was poured into ice-cold water and orange precipitate formed. The precipitate was filtered to give 2.8 g pure product as orange solid. ¹H NMR (CDCl₃) δ ppm: 3.92 (s, 3H), 3.99 (s, 3H), 7.07 (d, J=8.6 Hz, 2H), 7.33 (dd, J, =9.5 Hz, J₂=2.9 Hz, 1H), 7.46 (d, J=8.6 Hz, 1H), 7.60 (d, J=7.6 Hz, 1H), 7.73 (t, J=7.7 Hz, 1H), 7.97 (d, J=2.9 Hz, 1H), 8.28-8.40 (m, 3H), 9.91 (d, J=7.5 Hz, 1H); ¹³C NMR (CDCl₃) δ ppm: 55.42, 55.68, 109.07, 113.93, 122.46, 123.21, 124.80, 125.87, 126.36, 127.57, 128.71, 129.73, 129.93, 131.38, 131.54, 132.14, 133.87, 141.67, 159.39, 159.71; FD-MS: 366 (M⁺).

### Example 14

### Synthesis of Compound N

Compound M (2.2 g, 0.006 mol) was dissolved in 50 mL of methylene chloride and cooled to 0° C. To the solution was added titanium tetrachloride (1 M in methylene chloride, 7.2 mL) and borane-dimethylamine complex (0.85 g, 0.016 mol). Reaction turned dark green. After 20 min. the reaction was quenched with saturated sodium bicarbonate and extracted with methylene chloride. The crude product was purified by column chromatography on silica gel using methylene chloride/heptane as an eluent to give 1.2 g of pure product as off-white solid (56% solid). FD-MS: 352 (M⁺).

### Example 15

### Synthesis of Compound O

Compound M (1.2 g, 0.0034 mol) was dissolved in methylene chloride and cooled to 0° C. To the solution was added boron tribromide (1 M in methylene chloride, 30 mL) dropwise. The reaction was stirred for 4 h and quenched with saturated Na₂CO₃ solution, and extracted with ethyl acetate. The crude product was obtained as a 1.0 g brown solid (91% yield) and used without further purification. FD-MS: 324 (M⁺).

### Example 16

### Synthesis of Compound P

Compound O (1.0 g, 0.0028 mol) was dissolved in 20 mL of methylene chloride and triethylamine (0.86 g, 0.008 mol) was added. The reaction was cooled to 0° C. and triflic anhydride (2.4 g, 0.009 mol) was added slowly. The reaction was stirred at room temperature for a few hours. The reaction was quenched with water and extracted with ether and the crude product was purified by column chromatography on silica gel using methylene chloride/heptane as an eluent to give 1.2 g pure product as off-white solid (72% yield). FD-MS: 588 (M⁺).

### Synthesis of Polymers

### Example 17

### General Procedure for Synthesis of Polymers Via the Suzuki Coupling Reaction

Equal molar of aromatic di-bromide or di-triflate and aromatic di-boron compound, and phase transfer reagent Aliquat® 336 (0.10 equivalent to monomer) were dissolved in of toluene (the ratio of toluene to water (v/v) is about 3/1). To this solution was added 2 M Na₂CO₃ aqueous solution (3.3 equivalent to monomer). The reaction mixture was bubbled with dry nitrogen for 15 min and catalyst tetrakis(triphenylphosphine)palladium (0.03 equivalent to monomer) was added. The reaction was heated under vigorous reflux for 12-24 h, and small amount of phenylboronic acid was added for end-capping of bromo group. The reaction was heated for 5 h and bromobenzene was added to end-cap boronate group. The reaction was heated for another 4 h and then poured into 200 mL of methanol. The precipitated polymer washed with methanol, diluted HCl solution, and dried. The polymer was treated with diethyl dithiocarbamate twice: polymer was dissolved in toluene, and sodium diethyl dithiocarbamate in water (1 g in 10 mL of water) was added, and the mixture was stirred under nitrogen at 60° C. overnight. The toluene layer was separated and concentrated and the polymer was precipitated into methanol twice. Polymer can then be extracted with acetone with a Sohxlet setup overnight to remove oligomers. Polymer was dried under vacuum at 45° C.

### EL Device Fabrication and Performance

### Example 18

An EL device satisfying the requirements of the invention was constructed in the following manner. The organic EL medium has a single layer of the organic compound described in this invention.
a) An indium-tin-oxide (ITO) coated glass substrate was sequentially ultra-sonicated in a commercial detergent, rinsed with deionized water, degreased in toluene vapor and exposed to ultraviolet light and ozone for a few minutes.
b) An aqueous solution of PEDOT (1.3% in water, Baytron P Trial Product AI 4083 from H. C. Stark) was spin-coated onto ITO under a controlled spinning speed to obtain thickness of 500 Angstroms. The coating was baked in an oven at 110° C. for 10 min.
c) A toluene solution of a compound (300 mg in 30 mL of solvent) was filtered through a 0.2 μm Teflon filter. The solution was then spin-coated onto PEDOT under a controlled spinning speed. The thickness of the film was between 500-700 Angstroms.
d) On the top of the organic thin film was deposited a cathode layer consisting of 15 angstroms of a CsF salt, followed by a 2000 angstroms of a 10:1 atomic ratio of Mg and Ag.

The above sequence completed the deposition of the EL device. The device was then hermetically packaged in a dry glove box for protection against ambient environment.

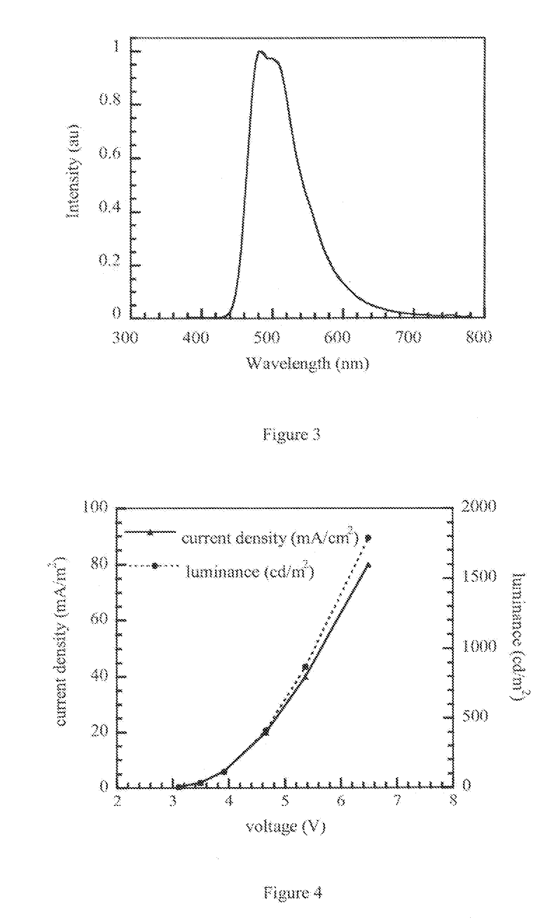

Table 1 summarizes the characterization of the polymers prepared in the present invention. Absorption (AB) and photoluminescence (PL) spectra were obtained from dilute solutions and solid thin films of the polymers and EL spectra were obtained from ITO/PEDOT/organic compound/CsF/Mg:Ag EL devices. The fabrication of EL devices was illustrated in example 18. FIG. 2 shows the absorption and PL spectra of compound 102. FIG. 3 shows the EL spectra of compound 102. And the voltage-current characteristics of the EL device of compound 102 is shown in FIG. 4.

TABLE 1
Characterization of polymers according to Examples.

| Com- | | | UV^{b} | PL^{c} | PL^{d} | EL |
|---|---|---|---|---|---|---|
| pound | M_{w}^{a} | PDI | (λₘₐₓ nm) | (λₘₐₓ nm) | (λₘₐₓ nm) | (λₘₐₓ nm) |
| 153 | 13300 | 2.19 | 409 | 459 (335) | 488 (360) | 516 |
| 102 | 10000 | 1.80 | 401 | 452 (325) | 478 (360) | 481 |
| 179 | 18000 | 2.51 | 409 | 454 (360) | 473 (360) | 496 |

^{a}weight average molecular weight, determined by size exclusion chromatography in THF using polystyrene standard.
^{b}in toluene solution
^{c}in toluene solution, the number in the parenthesis is the excitation wavelength.
^{d}solid state thin film, the number in the parenthesis is the excitation wavelength.

The invention has been described in detail with particular reference to certain preferred embodiments thereof, but it will be understood that variations and modifications can be effected within the spirit and scope of the invention.

### PARTS LIST

101 substrate
103 anode
105 hole-injecting layer
107 hole transporting layer
109 light emitting layer
111 electron-transporting layer
113 cathode
250 voltage-current source
260 electrical conductors

1. An organic material having a 6-member ring structure represented by the following formulae (I), 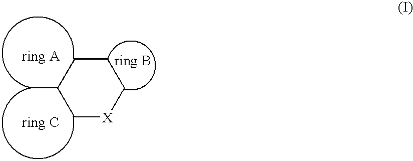
wherein:
ring A, ring B, and ring C each include substituted or un-substituted aromatic rings comprising 6 to 60 carbon atoms, or substituted or un-substituted heteroaromatic rings comprising 4 to 60 carbon atoms, and ring A and ring C form a fused aromatic or heteroaromatic structure; X is a carbon atom, a nitrogen atom, a sulfur atom, a silicon atom, an oxygen atom, a phosphorus atom, a selenium atom, or a germanium atom.

2. The organic material having a 6-member ring structure of claim 1 is a molecule or a polymer or mixture thereof.

3. The organic material having a 6-member ring structure of claim 1 is a molecule represented by formula (II)
(Y₁)y₁-formula (I)-(Y₂)y₂ (II)
wherein Y₁ and Y₂ are the same or different and each individually represent a substituted or unsubstituted alkyl, alkenyl, alkynyl, aryl, heteroaryl or alkoxy of 1 to 60 carbons, and y₁ and y₂ are integers from 0 to 6.

4. The organic material having a 6-member ring structure of claim 1 is a polymer represented by repeating units of formula (III) or (IV) 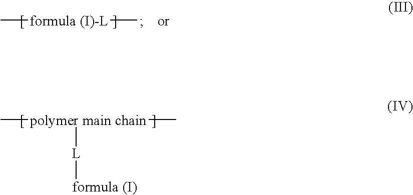
wherein:
L is a direct bond or a carbon linking group having 1 to 40 carbon atoms or a non-carbon linking group having 0 to 40 carbon atoms.

5. An electroluminescent material comprising one or more material having a 6-member ring structure of claim 1.

6. An electroluminescent device, comprising
a) a spaced-apart anode and cathode; and
b) an organic layer disposed between the spaced-apart anode and cathode and including a material of claim 1.

7. In an electroluminescent device comprising one or more active layers, wherein at least one of these active layers includes one or more organic material having a 6-member ring structure of claim 1.

8. A method of making an electroluminescent device, comprising:
a) providing a spaced-apart anode and cathode; and
b) depositing an organic layer between the spaced-apart anode and cathode and including a material of claim 1.

9. The organic material having a 6-member ring structure of claim 1 wherein ring A, ring B, and ring C each are substituted or un-substituted phenyl ring or pyrido ring.

10. The organic material having a 6-member ring structure of claim 1 where X is a carbon atom, a nitrogen atom, a sulfur atom, or an oxygen atom.

11. The organic material having a 6-member ring structure of claim 3 wherein ring A, ring B, and ring C each are substituted or un-substituted phenyl ring or pyrido ring.

12. The organic material having a 6-member ring structure of claim 3 where X is a carbon atom, a nitrogen atom, a sulfur atom, or an oxygen atom.

13. The organic material having a 6-member ring structure of claim 4 wherein ring A, ring B, and ring C each are substituted or un-substituted phenyl ring or pyrido ring.

14. The organic material having a 6-member ring structure of claim 4 where X is a carbon atom, a nitrogen atom, a sulfur atom, or an oxygen atom.

15. The organic material having a 6-member ring structure of claim 5 wherein ring A, ring B, and ring C each are substituted or un-substituted phenyl ring or pyrido ring.

16. The organic material having a 6-member ring structure of claim 5 where X is a carbon atom, a nitrogen atom, a sulfur atom, or an oxygen atom.

17. The organic material having a 6-member ring structure of claim 6 wherein ring A, ring B, and ring C each are substituted or un-substituted phenyl ring or pyrido ring.

18. The organic material having a 6-member ring structure of claim 6 where X is a carbon atom, a nitrogen atom, a sulfur atom, or an oxygen atom.

19. The organic material having a 6-member ring structure of claim 7 wherein ring A, ring B, and ring C each are substituted or un-substituted phenyl ring or pyrido ring.

20. The organic material having a 6-member ring structure of claim 7 where X is a carbon atom, a nitrogen atom, a sulfur atom, or an oxygen atom.

21. The organic material having a 6-member ring structure of claim 8 wherein ring A, ring B, and ring C each are substituted or un-substituted phenyl ring or pyrido ring.

22. The organic material having a 6-member ring structure of claim 8 where X is a carbon atom, a nitrogen atom, a sulfur atom, or an oxygen atom.
